(19) United States
(12) Patent Application Publication
(10) Pub. No.: US 2008/0275036 A1
(43) Pub. Date: Nov. 6, 2008

(54) Prevention and treatment of cardiac conditions

(75) Inventors: Maurice R. CROSS, Guemsey (GB); Stephen G. DILLY, Hillsborough, CA (US); Michael SCAIFE, Los Altos, CA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: APT Pharmaceuticals, Inc., Foster City, CA (US)

(21) Appl. No.: 11/799,825

(22) Filed: May 2, 2007

(51) Int. Cl. A61K 31/497; A61K 31/195; A61K 31/5375; A61P 9/00

(52) U.S. Cl. 514/230.8; 514/252.13; 514/254.05; 514/563

(57) ABSTRACT

The present invention provides a method of treating conditions associated with iron and calcium overload comprising administering an effective amount of dexrazoxane or a non-dexrazoxane compound of formula (IA), (IB), or (IC) or a pharmaceutically acceptable salt, tautomer, or stereoisomer thereof.

### REFERENCE TO RELATED APPLICATION

This application is related to PCT application ______, filed Nov. ______, 2005 claiming the priority to U.S. Provisional Patent Application Ser. No. 60/732,582. This PCT application is incorporated in its entirety herein.

### FIELD OF THE INVENTION

This invention relates to compositions and methods of preventing and treating cardiac conditions and other conditions relating to iron or calcium overload while minimizing undesirable side effects. The conditions treated include cardiotoxicity induced by environmental exposure or pharmacotherapy such as cancer chemotherapy.

### BACKGROUND

Dexrazoxane is known to prevent cardiomyopathy in cancer patients receiving high doses of the anthracycline agent, doxorubicin. To date, the mechanism by which dexrazoxane exerts its cardioprotective effect is not fully understood; however, it is suggested that an active metabolite, ADR-925, chelates iron accumulated in the heart from doxorubicin administration.

It is believed that calcium overload as well as iron and copper overload play a role in cardiotoxicity. Fibrillating atrial myocytes display signs of intracellular calcium overload, possibly because of rapid atrial depolarization and Ca2+-induced Ca2+ release from the sarcoplasmic reticulum (SR) stores. A thorough cytochemical analysis of calcium distribution in fibrillating goat atria has shown increased sarcolemma-bound and mitochondrial calcium deposits after 1 to 2 weeks of fibrillation. The increase in intracellular calcium in AF is associated with or followed by reduced accumulation of transcripts and protein for the sarcolemmal L-type Ca2+ channel and SR Ca2+ pump, whereas the expression of other SR proteins involved in calcium handling, such as calsequestrin, ryanodine receptor, and phospholamban, appears to be unaffected.

Dexrazoxane itself has undesirable properties that can limit its usefulness. Limited stability requires that the drug solution be prepared shortly before administration. The requirement of reconstitution at low pH and intravenous administration result in local toxicity at the site of administration. The distributional properties and pathway of metabolism can limit dexrazoxane's use in patients with compromised hepatic and renal capacity either from underlying disease states or from drug interactions. Dexrazoxane's intrinsic topoisomerase activity can result in toxicity to the patient. Accordingly, it is desirable to provide new compounds and compositions for, and methods of, preventing cardiotoxicity. The invention provides for compounds, compositions, and methods for preventing cardiotoxicity from environmental or pharmacological sources. Moreover, the compounds, compositions, and methods of the invention are useful in treating conditions associated with iron or calcium overload.

### SUMMARY

It is an object of this invention to provide an effective method of treating conditions associated with iron and calcium overload in a subject while minimizing undesirable side effects. Accordingly, the invention relates to a method of treating iron and calcium overload associated conditions including acute and chronic iron toxicity, polycythemia rubra vera, and typical cardiotoxicity resulting from cancer chemotherapy with agents such as anthracyclines. The minimization of side effects is generally, but not exclusively mediated by the administration of non-dexrazoxane compounds of the formula (IA), (IB), and (IC). Side effects associated with dexrazoxane administration for these and other purposes may be minimized by administering dexrazoxane in inhalation. Dexrazoxane is also provided for in the current invention for treating conditions associated with calcium cycling proteins. In particular, the mitigation, prevention, or remission of cardiotoxicity from epidermal growth factor receptor modulating drugs such as those used as cancer chemotherapeutic agents through the pretreatment and/or coadministration of dexrazoxane or the non-dexrazoxane compounds of the formula (IA), (IB), and (IC) is provided for. Heart failure, cardiac ischemia, hypoxia, and acute cardiac poisoning are conditions for which the administration of dexrazoxane or the non-dexrazoxane compounds of the formula (IA), (IB), and (IC) has utility in the present invention. Further, the invention provides for the prevention and treatment of atrial and ventricular arrhythmias in a mammal comprising the administration of a therapeutically effective dose of dexrazoxane, metabolites thereof (particularly ADR-925), and novel derivatives of dexrazoxane and their metabolites. The effects of dexrazoxane, metabolites thereof (particularly ADR-925), and novel derivatives of dexrazoxane and their metabolites through the newly discovered effects on calcium cycling proteins elucidate new opportunities for cardiac pharmacotherapy and represent the discovery of a new class (Class V) antiarrhythmic agents. The invention also provides for pharmaceutical compositions that may be formulated to achieve efficacy when administered to a patient but has increased stability or reduced compositional related adverse effects over the dexrazoxane compositions known in the art.

In a first embodiment, the invention provides a method of treating a condition arising from iron or calcium overload comprising administering to a patient in need thereof, an effective amount of a compound of formula (IA), (IB), or (IC):

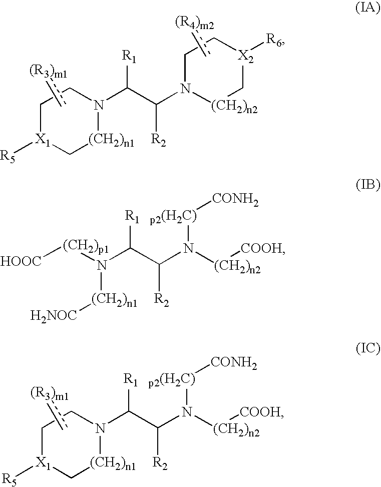

wherein,
X₁ and X₂ are independently selected form CH, N, S, or O;
each R₁ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl;
each R₂ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl; or
R₁ and R₂ are taken together to form a 3-7 membered substituted or unsubstituted carbocyclyl or heterocyclyl group;
each R₃ and R₄ group is independently selected from ═O, ═S, ═NH, H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl, wherein the dotted line represents the optional placement of a double bond; R₅ and R₆ are independently selected from H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl;
m1 and m2 are independently 1, 2, or 3; n1 and n2 are independently 0, 1, or 2; and p1 and p2 are independently 0, 1, or 2;
wherein if X₁ is O or S, then R₅ is absent, and wherein if X₂ is O or S, then R₆ is absent;
or a pharmaceutically acceptable salt, tautomer, stereoisomer, or prodrug thereof;
provided that if R₃ and R₄ are ═O, m1 and m2 are 2, and, if R₅ and R₆ are H, substituted or unsubstituted carbonyl, substituted or unsubstituted C₁₋₆ alkyl, substituted or unsubstituted C₁₋₆ alkoxy, or substituted or unsubstituted C₁₋₆ alkenyl, then R₁ and R₂ are not H, —CH₃, or taken together do not form —CH₂—, —CHCH—, or —CH₂CH₂—.

An embodiment provides for the treatment of a patient with impaired hepatic functioning with non-dexrazoxane compounds of formula (IA), (IB), and (IC). The methods are also useful in patient populations with genetic polymorphisms of a gene encoding for a calcium binding protein. The method also provides for the co-administration of a second or third drug compound including cancer chemotherapeutic agents. The agents can be anthracyclines such daunorubicin, doxorubicin, epirubicin, and idarubicin other cancer chemotherapeutic agents can be epidermal growth factor receptor (EGFR) modulating drugs including trastuzumab (Herceptin), cetuximab (Erbitux), bevacizumab (Avastin), panitumumab, gefitinib (Iressa), and erlotinib (Tarceva). In some embodiments of the invention the compounds are administered in a dosage ration that is less than about 25:1 of a compound according to formula (IA), (IB), or (IC) to the cancer chemotherapeutic agent.

In an embodiment of the invention, the method utilizes compounds with less toxicity and a higher therapeutic index than the cardioprotectant compound, dexrazoxane. The no observed adverse effect level (NOAEL) in this embodiment is typically 1.2 times or greater, preferably 1.5 time or greater than that of dexrazoxane. A further embodiment of the invention provides for compounds that may be formulated into non-IV dosage forms, particularly oral dosage forms such as tablets, capsules, suspensions, and sachets, from which the compounds according to formula (IA), (IB), or (IC) are orally bioavailable.

In a method according to the first embodiment, the compound has formula (IA). In a further related embodiment, X₁ and X₂ are N and n1 and n2 are 1. An additional embodiment provides for R₃ and R₄ are both being ═O, m1 and m2 are 2, and n1 and n2 are 1. In the above method, R₁ can be unsubstituted C₁₋₆ alkyl and R₂ is H. A further description of embodiment 1 is where the compound has formula (IC), R₂ is unsubstituted C₁₋₆ alkyl and R₁ is H. Additionally, in this embodiment, R₅ and R₆ can both be H. In an embodiment of formula (IB) or (IC), n1, n2, p1, and p2 are 1. Additionally, a particular embodiment provides for the case where R₃ and R₄ are appended to the adjacent (alpha) carbon to X₁ and X₂.

The invention provides for a second embodiment that is a method of treating a condition arising from iron or calcium overload comprising administering to a patient in need thereof, an effective amount of a compound of formula (IB):

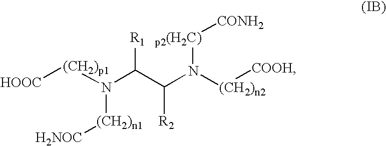

wherein,
R₁ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl;
R₂ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl; or
R₁ and R₂ are taken together to form a 3-7 membered substituted or unsubstituted carbocyclyl or heterocyclyl group;
n1 and n2 are independently 0, 1, or 2; and
p1 and p2 are independently 0, 1, or 2;
or a pharmaceutically acceptable salt, tautomer, stereoisomer, or prodrug thereof.

In a third embodiment of the invention, a pharmaceutical composition is provided for comprising a compound of formula (IA), (IB), or (IC):

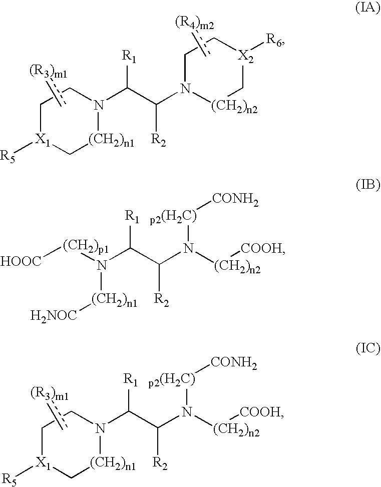

wherein,
X₁ and X₂ are independently selected form CH, N, S, or O;
each R₁ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl;
each R₂ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl; or
R₁ and R₂ are taken together to form a 3-7 membered substituted or unsubstituted carbocyclyl or heterocyclyl group;
each R₃ and R₄ group is independently selected from ═O, ═S, ═NH, H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl, wherein the dotted line represents the optional placement of a double bond; R₅ and R₆ are independently selected from H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl;
m1 and m2 are independently 1, 2, or 3; n1 and n2 are independently 0, 1, or 2; and p1 and p2 are independently 0, 1, or 2;
wherein if X₁ is O or S, then R₅ is absent, and wherein if X₂ is O or S, then R₆ is absent;
or a pharmaceutically acceptable salt, tautomer, stereoisomer, or prodrug thereof;
provided that if R₃ and R₄ are ═O, m1 and m2 are 2, and if R₅ and R₆ are H, substituted or unsubstituted carbonyl, substituted or unsubstituted C₁₋₆ alkyl, substituted or unsubstituted C₁₋₆ alkoxy, or substituted or unsubstituted C₁₋₆ alkenyl, then R₁ and R₂ are not H, —CH₃, or taken together do not form —CH₂—, —CHCH—, or —CH₂CH₂—; and
a pharmaceutically acceptable excipient.

A fourth embodiment provides for a method of treatment comprising administering to a patient in need thereof, an effective amount of a compound of formula (IA), (IB), or (IC):

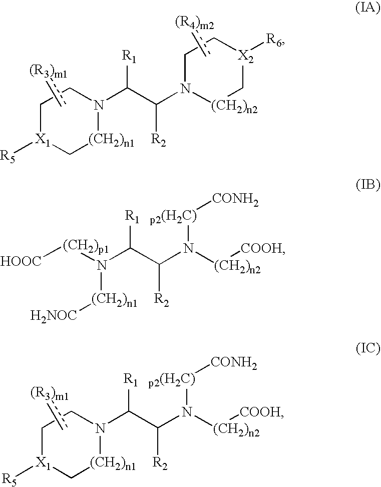

wherein,
X₁ and X₂ are independently selected form CH, N, S, or O;
each R₁ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl;
each R₂ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl; or
R₁ and R₂ are taken together to form a 3-7 membered substituted or unsubstituted carbocyclyl or heterocyclyl group;
each R₃ and R₄ group is independently selected from ═O, ═S, ═NH, H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl, wherein the dotted line represents the optional placement of a double bond; R₅ and R₆ are independently selected from H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl;
m1 and m2 are independently 1, 2, or 3; n1 and n2 are independently 0, 1, or 2; and p1 and p2 are independently 0, 1, or 2;
wherein if X₁ is O or S, then R₅ is absent, and wherein if X₂ is O or S, then R₆ is absent;
or a pharmaceutically acceptable salt, tautomer, stereoisomer, or prodrug thereof, and an EGFR modulating agent. The embodiment provides for the EGFR modulating agent being trastuzumab, cetuximab, bevacizumab, panitumumab, gefitinib, or erlotinib. In a further embodiment of the above method, non-dexrazoxane compounds of formula (IA), (IB), and (IC) are used which exhibit less toxicity and a higher therapeutic index than the cardioprotectant compound, dexrazoxane. The no observed adverse effect level (NOAEL) in this embodiment is typically 1.2 times or greater, preferably 1.5 time or greater than that of dexrazoxane. A further embodiment of the invention provides for compounds that may be formulated into non-IV dosage forms, particularly oral dosage forms such as tablets, capsules, suspensions, and sachets, from which the compounds according to formula (IA), (IB), or (IC) are orally bioavailable.

A fifth embodiment related to the use of dexrazoxane for a method for the treatment and prevention of cardiotoxicity associated with the administration of epidermal growth factor receptor modulating agents including trastuzumab (Herceptin), cetuximab (Erbitux), bevacizumab (Avastin), panitumumab, gefitinib (Iressa), and erlotinib (Tarceva) by the administration of an effective amount of dexrazoxane. The use of dexrazoxane in this and other embodiments is of particular use in treating patients who are susceptible to cardiotoxicity due to a genetic polymorphism involving a calcium cycling protein, i.e., SR Ca2+ cycling proteins, such as phospholamban and S100A1. Accordingly an embodiment of the invention is the use of dexrazoxane and non-dexrazoxane compounds of formula (IA), (IB), and (IC) for the treatment or prevention of cardiotoxicity in patients with a genetic polymorphism of a calcium cycling protein gene who are undergoing treatment with a cancer chemotherapy agent, particularly an EGFR modulating cancer chemotherapy agent such as trastuzumab, cetuximab, bevacizumab, panitumumab, gefitinib, and erlotinib.

In a sixth embodiment, the invention related to a method of reversing or preventing QT interval prolongation by administering an effective amount of dexrazoxane or non-dexrazoxane compounds of formula (IA), (IB), and (IC) to a patient. The patient groups benefiting from such therapy include patients with prolonged QT syndrome, drug induced QT prolongation, patients undergoing anesthesia, and patients experience Torsade de pointes. This condition can be particularly critical in patients with genetic polymorphism of calcium cycling proteins and also those treated with EGFR activity modulating agents such as the anti-EGFR cancer chemotherapeutic drugs.

In a seventh embodiment, the invention relates to a method of treatment of a disease or condition while minimizing adverse effects through the administration of dexrazoxane and non-dexrazoxane compounds of formula (IA), (IB), and (IC) via inhalation. Delivered to the heart in this manner, the dexrazoxane or non-dexrazoxane compound of the formula (IA), (IB), or (IC) is presented directly at the desired site of action to exert a desired cardiac effect. The desired effect can be cardioprotective, antiarrhythmic, anti-QT prolongation, or other additional cardiac effects as described in the present application. By delivering the drug agent directly to the heart, the required efficacious dose is generally lower. Delivery of dexrazoxane or a non-dexrazoxane compound of the formula (IA), (IB), or (IC) to the heart via inhalation results in reduced systemic exposure and lowered toxicity or lowered incident of adverse effects. Administration of the subject compounds by inhalation can achieve high levels of compounds at the site of action at a faster rate thereby achieving a more rapid onset of action. The inhalation route of administration of the subject compounds results in an improved ratio of cardiac to systemic exposure. In a particular aspect of this embodiment, a solution of dexrazoxane for aerosolization is provided. In another aspect, a dry powder formulation of dexrazoxane is provided. This embodiment also provides for the use of dexrazoxane or a non-dexrazoxane compound of formula (IA), (IB), or (IC) for the manufacture of a medicament for administration via inhalation. Also provided for is the use of dexrazoxane or a non-dexrazoxane compound of formula (IA), (IB), or (IC) for the treatment or prevention of cardiotoxicity, arrhythmia, QT-prolongation, cardiac failure, or other conditions of iron or calcium overload.

In a method according to the third and fourth embodiments, the compound has formula (IA). In a further related embodiment, X₁ and X₂ are N and n1 and n2 are 1. An additional embodiment provides for R₃ and R₄ are both being ═O, m1 and m2 are 2, and n1 and n2 are 1. In the above method, R₁ can be unsubstituted C₁₋₆ alkyl and R₂ is H. A further description of the third and fourth embodiments is where the compound has formula (IC), R₂ is unsubstituted C₁₋₆ alkyl and R₁ is H. Additionally, in this embodiment, R₅ and R₆ can both be H. In an embodiment of formula (IB) or (IC), n1, n2, p1, and p2 are 1. Additionally, a particular embodiment provides for the case where R₃ and R₄ are appended to the adjacent (alpha) carbon to X₁ and X₂.

The present invention also encompasses pharmaceutical compositions of each of the compounds described herein.

Methods of manufacturing the compositions described herein are provided and contemplated to fall within the scope of the invention, as is the use of the compositions in methods for manufacturing medicaments for use in the methods of the invention.

Further embodiments of the invention include those described in the detailed description.

### DESCRIPTION OF THE FIGURES

FIG. 1. Effects of Dexrazoxane on developed tension in guinea pig whole hearts.

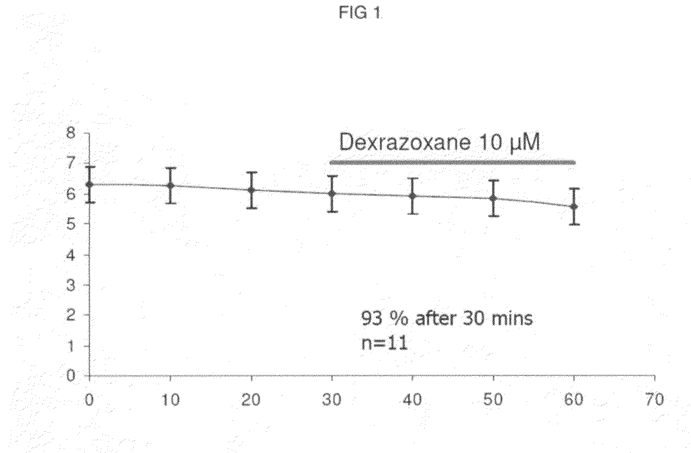

FIG. 2. Effects of Dexrazoxane on unloaded cell shortening in single ventricular myocytes.

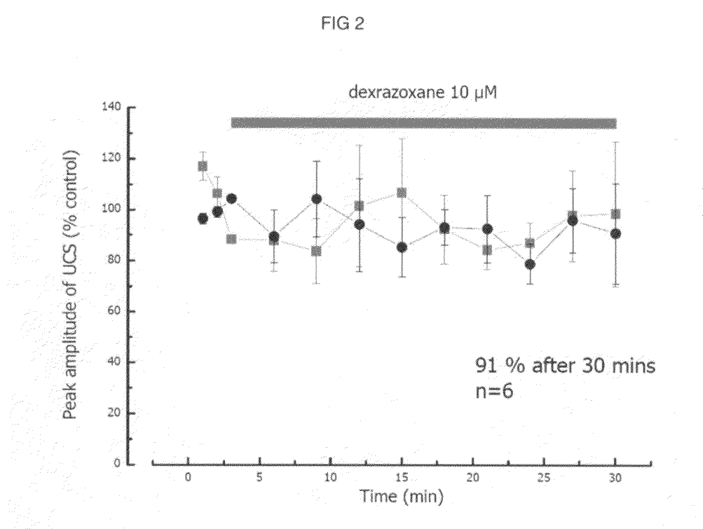

FIG. 3. Mean data showing the effect of dexrazoxane (10 μM) on action potential duration (APD₉₀) is shown. The data shows that perfusion with dexrazoxane (27 minutes) caused an 18±3% shortening of APD₉₀ from 212±9 ms to 168±12 ms (n=4, P<0.005).

FIG. 4. The addition of dexrazoxane to the superfusion solution caused an 18±4% a decrease in APD₄₀ from 175±8 ms to 136±15 ms (n=3, P<0.005).

FIG. 5. Superfusion of dexrazoxane did not significantly alter the APD₉₀₋₄₀ duration which is taken as an index of triangulation. Accordingly, time control data show that under control condition there was no change in either APD₉₀ (FIG. 3), APD₄₀ (FIG. 4), or in APD₉₀₋₄₀ (FIG. 5).

FIG. 6. Mean data for the peak amplitude of action potential driven unloaded cell shortening (UCS) measured in single ventricular myocytes is shown. Application of dexrazoxane (10 μM, 27 minutes) did not significantly alter the amplitude of UCS expressed as absolute cell shortening (control, 9.4±3.4 μm; dexrazoxane 10.7±3.2 μm, n=3, P=ns; the left panel of FIG. 6 shows contraction in μm while the right panel shows percentage change from control).

FIG. 7. Similar findings to FIG. 6 were made when shortening was expressed as percentage of resting cell length as in FIG. 7 (control, 8.5±2.7% RCL; dexrazoxane 8.0±2.0% RCL, n=3, P=ns; the left panel of FIG. 7 shows shortening as percentage of cell length while the right panel shows percentage change from control).

FIG. 8. Effects of Dexrazoxane on APD₉₀ in single ventricular myocytes. FIG. 8 shows typical action potential records taken from a single ventricular myocytes under control conditions and in the presence of dexrazoxane (10 μM) superfused for 10 minutes, 20 minutes and 30 minutes. Dexrazoxane (under these conditions) shortened action potential duration.

FIG. 9. Effects of Dexrazoxane on APD₉₀ and QT interval.

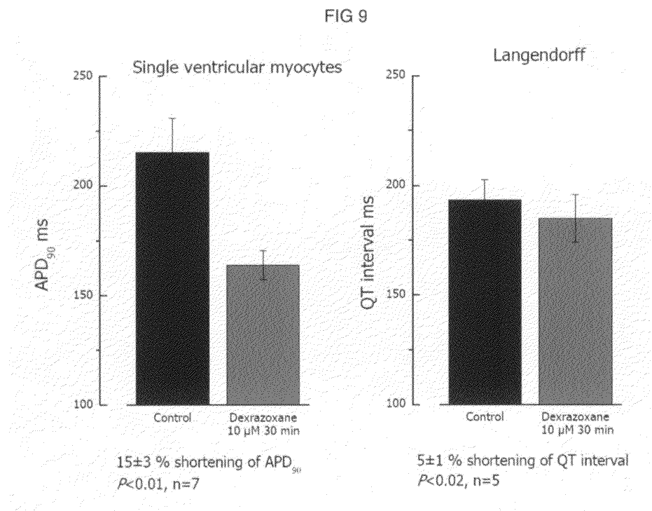

FIG. 10. Effects of Dexrazoxane on heart rate variability in stable Langendorff heart preparations

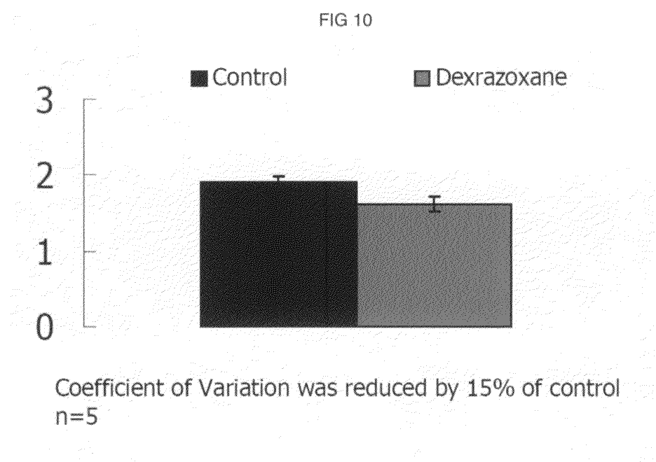

FIG. 11. Effects of Dexrazoxane on heart rate variability in unstable Langendorff heart preparations

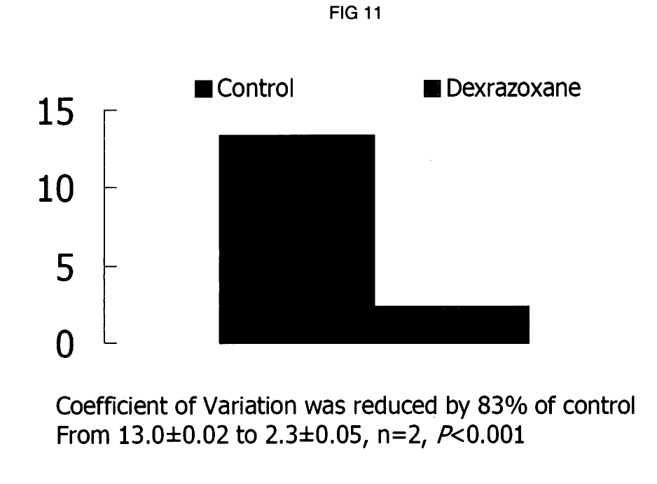

FIG. 12. Effects of total flow occlusion on heart rate variability in stable Langendorff heart preparations. In the presence of dexrazoxane (10 μM, 30 min) the coefficient of variation was increased by only 77% during reperfusion following a 15 min period of total flow ischemia (from control, 2.9±0.2 to reperfusion 3.3±1.1 n=3, P=ns).

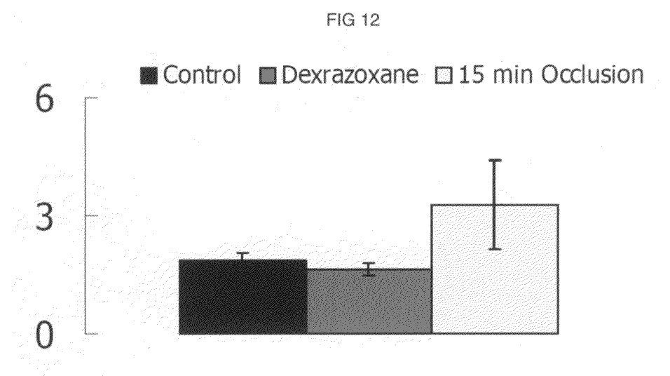

FIG. 13. (a) Metabolites formed from dexrazoxane (B, C, and ADR-925); (b) catalyzed by dihydroorotase (DHOase); (c) which is inhibited by furosemide.

FIG. 14. This figure shows the effects of 10 uM dexrazoxane, 30 uM dexrazoxane, and 100 uM dexrazoxane versus control on calcium flux (top) and action potential duration (bottom) in isolated guinea pig myocytes.

FIG. 15. This figure shows the effects of 30 uM doxorubicin, 100 uM doxorubicin, and 300 uM doxorubicin versus control on calcium flux (top) and action potential duration (bottom) in isolated guinea pig myocytes.

### DETAILED DESCRIPTION

The present invention provides compounds, compositions, and methods of treating conditions of iron and calcium overload including cancer chemotherapy induced cardiotoxicity.

Conditions associated with iron and calcium overload include acute iron toxicity and toxicity associated with chronic iron exposure. The vast majority of childhood iron poisonings are unintentional and result in no or minimal toxicity. The most serious exposures involve prenatal vitamins and pure iron preparations that contain ferrous sulfate tablets, which typically have significantly more elemental iron per tablet (60 to 65 mg) than other iron preparations. Iron may also accumulate in the body if a person is given iron therapy in excessive amounts or for too long, with repeated blood transfusions and in chronic alcoholism. Acute iron toxicity causes effects on the GI tract and the cardiovascular, metabolic, hepatic, and central nervous systems. Once excess iron has been ingested, the body has no natural means of getting rid of it so it continues to store it, as it would do with iron that is ingested in normal circumstances through the diet. It is stored in the reticulo-endothelial cells of the liver as well as in other organs, and these organs are the most affected by a large intake of iron. Ingestion of iron is also dangerous as in solution it is corrosive properties, which damage the GI tract. It is also readily absorbed by this route and enters the circulation. Excess free iron is a mitochondrial toxin that leads to derangements in energy metabolism. Although iron poisoning is a clinical diagnosis, serum iron levels are useful in predicting the clinical course of the patient.

The absorption of iron is normally very tightly controlled by the GI system. However, in overdose, local damage to the GI mucosa allows unregulated absorption, which leads to potentially toxic serum levels.

Much of the pathophysiology of iron poisoning is a result of metabolic acidosis and its effect on multiple organ systems. Toxicity manifests as local and systemic effects. Typically, iron poisoning is described in 5 sequential phases. No universal agreement exists as to the number of phases or the times assigned to those phases. Patients may not always demonstrate all of the phases.

Phase 1, initial toxicity, predominantly manifests as GI effects. This phase begins during the first 6 hours postingestion and is associated with hemorrhagic vomiting, diarrhea, and abdominal pain. This is predominantly due to direct local corrosive effects of iron on the gastric and intestinal mucosa. Early hypovolemia may result from GI bleeding, diarrhea, and third spacing due to inflammation. This can contribute to tissue hypoperfusion and metabolic acidosis.

Convulsions, shock, and coma may complicate this phase if the circulatory blood volume is sufficiently compromised. In these cases, the patient progresses directly to phase 3, possibly within several hours.

Phase 2 is known as the latent phase and typically occurs 4-12 hours postingestion. It is usually associated with an improvement in symptoms, especially when supportive care is provided during phase 1. During this time, iron is absorbed by various tissues, and systemic acidosis increases. Clinically, the patient may appear to improve, especially to nonmedical personnel, because the vomiting that occurs in phase 1 subsides. However, laboratory analysis demonstrates progressive metabolic acidosis and, potentially, the beginning of other end-organ dysfunction (ie, elevation of transaminase levels).

Phase 3 typically begins within 12-24 hours postingestion, although it may occur within a few hours following a massive ingestion. Following absorption, ferrous iron is converted to ferric iron, and an unbuffered hydrogen ion is liberated. Iron is concentrated intracellularly in mitochondria and disrupts oxidative phosphorylation, resulting in free radical formation and lipid peroxidation. This exacerbates metabolic acidosis and contributes to cell death and tissue injury at the organ level.

Phase 3 consists of marked systemic toxicity caused by this mitochondrial damage and hepatocellular injury. GI fluid losses lead to hypovolemic shock and acidosis. Cardiovascular symptoms include decreased heart rate, decreased myocardial activity, decreased cardiac output, and increased pulmonary vascular resistance. The decrease in cardiac output may be related to a decrease in myocardial contractility exacerbated by the acidosis and hypovolemia. Free radicals from the iron absorption may induce damage and play a role in the impaired cardiac function.

The systemic iron poisoning in phase 3 is associated with a positive anion gap metabolic acidosis. The following explanations for the acidosis have been proposed: Conversion of free plasma iron to ferric hydroxide is accompanied by a rise in hydrogen ion concentration; free radical damage to mitochondrial membranes prevents normal cellular respiration and electron transport, with the subsequent development of lactic acidosis; hypovolemia and hypoperfusion contribute to acidosis, and cardiogenic shock contributes to hypoperfusion.

A coagulopathy is observed and may be due to 2 different mechanisms. Free iron may exhibit a direct inhibitory effect on the formation of thrombin and thrombin's effect on fibrinogen in vitro. This may result in a coagulopathy. Later, reduced levels of clotting factors may be secondary to hepatic failure.

Phase 4 may occur 2-3 days postingestion. Iron is absorbed by Kupffer cells and hepatocytes, exceeding the storage capacity of ferritin and causing oxidative damage. Pathologic changes include cloudy swelling, periportal hepatic necrosis, and elevated transaminase levels. This may result in hepatic failure.

Phase 5 occurs 2-6 weeks postingestion and is characterized by late scarring of the GI tract, which causes pyloric obstruction or hepatic cirrhosis.

Cardiotoxicity is associated with some cancer chemotherapies and can be limiting to continued treatment and can be related to localized iron or calcium overload in the patient. Cardiotoxicity includes reversible toxicity and irreversible toxicities. An example of a reversible toxicity would be the prolongation of QT interval due to environmental (including poisoning or dietary means) or pharmacological toxicities that returns to a normal or near normal state without pathology upon resolution of the toxic episode. Irreversible cardiotoxicity arises when damage to the heart occurs environmentally (including poisoning or through dietary means) or pharmacologically which is not resolved by removal of the source of the toxicity and which results in a long term or permanent pathology or cardiomyopathy.

The compounds and compositions of the invention are useful in the treatment of toxicity from acute and chronic iron exposure. The compounds and compositions are used for treating localized iron or calcium overload in the case of environmentally mediated or pharmacologically mediated cardiotoxicity.

An embodiment of the invention provides for the use of dexrazoxane pharmaceutical compositions and the other pharmaceutical compositions comprising the compounds of formula (IA), (IB), and (IC) for the prevention of cardiotoxicity in patients receiving cancer therapeutics acting through the epidermal growth factor receptor (EGFR) and, in a further embodiment, the Her1/Her2/Her3/Her4 pathways. Exemplary chemotherapeutic agents for which the dexrazoxane compositions and for which the pharmaceutical compositions comprising compounds formula (IA), (IB), and (IC) can be used in combination for the prevention of cardiotoxicity include trastuzumab (Herceptin), cetuximab (Erbitux), bevacizumab (Avastin), panitumumab, gefitinib (Iressa), erlotinib, (Tarceva). In another embodiment, dexrazoxane pharmaceutical compositions and the other pharmaceutical compositions comprising compounds of formula (IA), (IB), and (IC) are useful for the prevention of cardiotoxicity in a subpopulation of patients which are susceptible to cardiotoxicity when treated with cancer therapeutics acting through epidermal growth factor receptor (egrf) (and Her1/Her2/Her3/Her4) pathways including trastuzumab (Herceptin), cetuximab (Erbitux), bevacizumab (Avastin), panitumumab, gefitinib (Iressa), erlotinib, (Tarceva). In a further embodiment, the susceptible patient subpopulation comprise individuals with a calcium binding protein polymorphism. The compounds of formula (IA), (IB), and (IC) are also useful in preventing cardiotoxicity of anthracyclines such as daunorubicin, doxorubicin, epirubicin, and idarubicin. The use of anti-EGFR chemotherapy agents in combination with anthracycline cancer chemotherapy agents can result in synergistic cardiotoxicity. The use of dexrazoxane and non-dexrazoxane compounds of formula (IA), (IB), and (IC) are of particular use in treating or preventing the synergistic cardiotoxicity arising from combination therapy of EGFR modulating agents (anti-EGFR agents including trastuzumab, cetuximab, bevacizumab, panitumumab, gefitinib, and erlotinib) and anthracycline cancer chemotherapeutic agents (including daunorubicin, doxorubicin, epirubicin, and idarubicin).

The use of dexrazoxane is provided for in a method for the treatment and prevention of cardiotoxicity associated with the administration of epidermal growth factor receptor modulating agents including trastuzumab, cetuximab, bevacizumab, panitumumab, gefitinib, and erlotinib by the administration of an effective amount of dexrazoxane. The use of dexrazoxane in this and other embodiments is of particular use in treating patients who are susceptible to cardiotoxicity due to a genetic polymorphism involving a calcium cycling protein, i.e., SR Ca2+ cycling proteins, such as phospholamban and S100A1. Accordingly an embodiment of the invention is the use of dexrazoxane and non-dexrazoxane compounds of formula (IA), (IB), and (IC) for the treatment or prevention of cardiotoxicity in patients with a genetic polymorphism of a calcium cycling protein gene who are undergoing treatment with a cancer chemotherapy agent, particularly an EGFR modulating cancer chemotherapy agent such as trastuzumab, cetuximab, bevacizumab, panitumumab, gefitinib, and erlotinib. Calcium movement is central to the process of excitation-contraction coupling in cardiac myocytes. Membrane depolarization results in the entry of calcium via the L-type calcium channel. This triggers release of intracellular stores of calcium from the sarcoplasmic reticulum (SR) via the cardiac ryanodine receptors (RYR), which are calcium-release channels. The resulting rise in intracellular calcium causes myofilament activation and muscle contraction. Conversely, a fall in intracellular calcium initiates relaxation. Two factors drive the drop in intracellular Ca2+ that initiates relaxation: reuptake into the SR via SR Ca2+-ATPase (SERCA2a) and the pumping of Ca2+ out of the cell in exchange for Na+ via the sodium-calcium exchanger (NCX). Phospholamban, which in its unphosphorylated state inhibits SERCA2a, influences the Ca2+-pumping activity of SERCA2a. The expression levels of the SR Ca2+-handling proteins in normal and failing human hearts may differ, since their depressed cardiac function has been suggested to reflect abnormalities at the sarcoplasmic reticulum level. Levels of SR Ca2+ cycling proteins, such as phospholamban relative to the SR Ca2+-ATPase are increased in human heart failure. This increase may constitute a contributing factor to diastolic dysfunction and depressed contractility exhibited by the failing heart. Furthermore, decreased phosphorylation of PLN, without alterations in its protein levels, has been suggested to contribute to the depressed function in human heart failure. The attenuated phospholamban phosphorylation is at least partially due to increased protein phosphatase-1 activity, reflecting inactivation of its Inhibitor-1 protein in human failing hearts. Indeed, both Inhibitor-1 and phospholamban phosphorylation are decreased in human heart failure. Naturally occurring mutations in the SR Ca2+-ATPase, phospholamban and Inhibitor-1 genes may alter SR Ca2+ cycling and contractility, leading to remodeling and heart failure. The genetic polymorphisms that influence the Ca2+-pumping activity may include naturally occurring mutation(s) in the human phospholamban gene. A truncation mutation (L39stop) has been identified in individuals from two families with inherited dilated cardiomyopathy that resulted in dramatically diminished myocardial phospholamban protein content, consequent loss of phospholamban inhibition of SR Ca2+-ATPase, and the development of heart failure and early mortality in homozygous individuals. In contrast to mice in which phospholamban-deficiency enhances myocardial inotropy and lusitropy without adverse effects, phospholamban is essential for cardiac health in humans and its absence results in lethal heart failure. Additional mutations may include mutations at the 5′ and 3′ UTR of the phospholamban gene. By identifying patients with calcium cycling gene polymorphisms that could affect cardiac functioning, the patients could be treated by the subject compounds and compositions of the invention before cardiotoxicity arose due to pharmacotherapy such as cancer chemotherapy.

The pharmaceutical compositions of the present application comprising compounds of formula (IA), (IB), and (IC) can be used in clinical scenarios where the use of dexrazoxane compositions are contraindicated or are otherwise undesirable. In one embodiment, the compositions are formulated in intravenous or intramuscular preparations at less acidic pH than dexrazoxane IV preparations. The less acidic formulations comprising compounds of formula (IA), (IB), and (IC) have less local toxicity at the site of injection and, in the case of IV formulations, less toxicity to the vein that is used for administration. Such toxicities that are reduced may include inflammation, necrosis, granuloma, extravasion, pain, vessel occlusion, etc. The local toxicity of the compositions may be compared to dexrazoxane IV compositions in humans using the Rittenberg scale or through other in vivo models including, for example, the mouse writhing assay. In one aspect of the invention, the IV or IM formulations of compounds of formula (IA), (IB), and (IC) have a 20% or greater, a 25% or greater, a 30% or greater or a 35% or greater reduction in toxicity. In a further aspect of the invention, the IV or IM formulations of compounds of formula (IA), (IB), and (IC) have a 40% or greater, a 45% or greater, a 50% or greater, a 75% or greater, an 85% or greater, or a 95% or greater reduction in toxicity.

In clinical use, dexrazoxane is reconstituted prior to administration and is stable for 6 hours. The IV or IM formulations comprising compounds of formula (IA), (IB), and (IC) have improved stability over dexrazoxane formulations. In an embodiment of the invention, the formulations are stable in solution for 12 hours or greater. In another embodiment of the invention, the formulations are stable for 24 hours or greater. In a further embodiment of the invention, the formulations are stable for 48 hours or greater. In a further embodiment of the invention, the formulations are stable for 168 hours or greater. In a further embodiment, the formulations are stable for 30 days or greater.

The pharmaceutical compositions comprising compounds of formula (IA), (IB), and (IC) may be formulated as stable oral dosage forms. In such form, dosing regimens outside of those known for dexrazoxane are facilitated. The compounds of formula (IA), (IB), and (IC) can exhibit reduced systemic toxicity, reduced mutagenicity, reduced teratogenicity, and reduced clastogenicity as compared to dexrazoxane or dexrazoxane metabolites. The compounds of formula (IA), (IB), and (IC) (excluding dexrazoxane) can exhibit reduced myelotoxicity, leucopenia and thrombocytopenia and produce less hepatic and renal impairment than dexrazoxane or dexrazoxane metabolites. These reduced effects are measurable as an increased no observed adverse effect level (NOAEL) in toxicological models. In an embodiment of the invention, the compounds of formula (IA), (IB), and (IC) (excluding dexrazoxane) exhibit a NOAEL that is 1.2× or greater than that of dexrazoxane. In an additional embodiment, the NOAEL is 1.5× or greater than that of dexrazoxane. In a further embodiment, the NOAEL is 2× or greater than that of dexrazoxane. In a further embodiment of the invention, the NOAEL is 3× or greater than that of dexrazoxane. In still a further embodiment, the NOAEL is 5× or greater than that of dexrazoxane. The reduced toxicity of the compounds of formula (IA), (IB), and (IC) (excluding dexrazoxane) result in a greater therapeutic index and the opportunity to treat with doses that achieve greater efficacy and to use dosing schedules that facilitate prolonged exposure levels, better loading of the cardioprotective compound, and chronic use of the pharmaceutical compositions. In an embodiment of the invention, the enhanced safety of pharmaceutical compositions comprising a compound of formula (IA), (IB), and (IC) (excluding dexrazoxane) is due to reduced systemic exposure to dexrazoxane. The compounds of the formula (IA), (IB), and (IC) (excluding dexrazoxane) can partition into the heart more readily than dexrazoxane where they act directly and through metabolic activation in the myocyte to exert their cardioprotective effects. These properties make the pharmaceutical compositions comprising a compound of the formula (IA), (IB), and (IC) (excluding dexrazoxane) safer and more efficacious in individuals with decreased hepatic function. Additionally, the invention includes a method for the treatment or prevention of cancer chemotherapeutic induced cardiotoxicity with reduced interference with the efficacy of the cancer chemotherapeutic by administering an effective amount of a pharmaceutical composition comprising a compound of the formula (IA), (IB), and (IC) (excluding dexrazoxane). In an embodiment of this method, a pharmaceutical composition comprising a compound of the formula (IA), (IB), and (IC) (excluding dexrazoxane) is administered prior and/or during treatment with an anthracycline cancer therapeutic agent wherein the efficacy of the anthracycline cancer therapeutic agent is inhibited less that the inhibition associated with administration of dexrazoxane. In practicing this method, the anthracycline agent may be daunorubicin, doxorubicin, epirubicin, and idarubicin.

In a method of the invention, cardiotoxicity is reduced when pharmaceutical compositions comprising an effective amount of compounds of formula (IA), (IB), and/or (IC) are administered once, twice, three times, four times, or five times daily for 1 day, 2 days, 3 days, 4 days, 5 days, 6 days, or 7 days or greater prior to the administration of a pharmaceutical agent that produces cardiotoxicity. The dosing may be terminated prior to or may be continued through the course of treatment with a pharmaceutical agent that produces cardiotoxicity. During dosing concomitant with the cardiotoxic pharmaceutical agent, pharmaceutical compositions comprising compounds of formula (IA), (IB), and/or (IC) are dosed once, twice, three times, four times, or five times daily. The administration of the compositions may be stopped when the administration cardiotoxic pharmaceutical agent is stopped or the administration of the compositions may be continued until the cardiotoxic pharmaceutical agent has been substantially cleared from the subject's body.

In an embodiment of the present invention, a pharmaceutical composition comprising a compound of formula (IA), (IB), or (IC) is administered chronically, with or without chronic maintenance therapy of a cardiotoxic pharmaceutical agent.

The non-dexrazoxane compounds of formula (IA), (IB), and/or (IC) can exhibit enhanced potency over that of dexrazoxane and exhibit a greater therapeutic index. One skilled in the art can readily determine a safe and effective dose of the pharmaceutical compositions described herein. The loading dose of any given compound of the present invention will depend on the potency of the drug compound, the bioavailability of the drug compound, and the volume of distribution of the drug compound. A typical loading dose may be in the range of about 0.25 to about 30 mg drug compound per kg patient weight. A preferred loading dose is from about 1 mg/kg to about 28 mg/kg. Another embodiment of the invention provides for a loading dose from about 1 mg/kg to about 25 mg/kg. A further embodiment of the invention provides for a loading dose from about 5 mg/kg to about 22 mg/kg. A further embodiment of the invention provides for a loading dose from about 10 mg/kg to about 20 mg/kg. Wherein the safety of the non-dexrazoxane compound of formula (IA), (IB), and (IC) exceeds that of dexrazoxane, it may be advantageous to administer higher doses of the subject compound(s). In this situation, a typical loading dose may be in the range of about 25 to about 200 mg drug compound per kg patient weight. A preferred loading dose is from about 30 mg/kg to about 175 mg/kg. Another embodiment of the invention provides for a loading dose from about 40 mg/kg to about 150 mg/kg. A further embodiment of the invention provides for a loading dose from about 50 mg/kg to about 100 mg/kg. A further embodiment of the invention provides for a loading dose from about 75 mg/kg to about 150 mg/kg. Maintenance doses may be administered following the loading dose to ensure that therapeutic concentrations of the non-dexrazoxane compounds of formula (IA), (IB), and (IC) are maintained at the site of action. The maintenance dose may be determined by the pharmacokinetic properties mentioned above for the particular compounds of the present invention but will also be determined, in part, by the clearance of the particular compound.

When used to prevent cardiotoxicity associated with cancer therapeutic agents, the non-dexrazoxane compounds of formula (IA), (IB), and (IC) (cardiotoxicity preventing compounds) are administered in a dosage ratio to that of the cancer therapeutic agent are less than about 50:1, less than about 40:1, less than about 25:1, less than about 15:1, less than about 10:1, less than about 5:1, less than about 2.5:1, less than about 1:1, less than about 0.5:1, or less than about 0.1:1. The non-dexrazoxane compounds of formula (IA), (IB), and (IC) are administered in a dosage ratio to that of the cancer therapeutic agent of from about 50:1 to about 0.01:1. In an embodiment, the cardiotoxicity preventing compound to cancer therapeutic agent ratio is from about 40:1 to about 0.1:1. In a further embodiment, the cardiotoxicity preventing compound to cancer therapeutic agent ratio is from about 30:1 to about 0.5:1. In a further embodiment, the cardiotoxicity preventing compound to cancer therapeutic agent ratio is from about 20:1 to about 0.5:1. In a further embodiment, the cardiotoxicity preventing compound to cancer therapeutic agent ratio is from about 10:1 to about 0.5:1. In other embodiments, cardiotoxicity preventing compound to cancer therapeutic agent ratio is about 10:1, about 5:1, about 3:1, about 2:1, about 1:1, or about 0.5:1. When used to prevent cardiotoxicity associated with cancer therapeutic agents, the non-dexrazoxane compounds of formula (IA), (IB), and (IC) (cardiotoxicity preventing compounds) may be administered in a range of from about 1 to about 2500 mg/m², from about 5 to about 2000 mg/m², from about 10 to about 1500 mg/m², from about 20 to about 1500 mg/m², or from about 35 to about 1000 mg/m².

In an embodiment of the invention, the maintenance dose is tailored to the particular patient based on patient weight and, optionally, on the clearance of the compound. Clearance of the compounds of formula (IA), (IB), and (IC) may be affected by drug-drug interactions. In particular, inhibitors of the p-glycoprotein pump (Pgp) may decrease the clearance of the compounds of formula (IA), (IB), and (IC). In one embodiment of the invention, the compounds are co-administered with a Pgp inhibitor. Examples Pgp inhibitors include ketoconazole, erythromycin, verapamil, quinidine, probenecid and cimetidine.

The successful application of dexrazoxane or a pharmaceutically acceptable salt, derivative, tautomer, stereoisomer, or metabolite thereof to the treatment of conditions associated with iron and calcium overload including cardiotoxicity is evidenced by the evaluation of the thermodynamic properties of the compound, e.g., measuring its partition coefficient between water and octanol, evaluation of its kinetics of elimination by measuring its stability in buffer and in human plasma, and evaluation of its safety and physiological properties in humans and animal preparations. More specifically, dexrazoxane can be used for preventing life-threatening cardiotoxicity, especially in patients with undergoing cancer chemotherapy. Compositions comprising the compounds of formula (IA), (IB), and (IC) have many advantages over the current IV formulation of dexrazoxane including more rapid onset of action, increased efficacy, decreased toxicities, new dosing regimens permitting long term exposure and greater propensity for adherence, lower topoisomerase associated toxicities.

In addition, the compounds can be included in a composition comprising a second active ingredient. The second active ingredient can be useful for concurrent or synergistic prevention of cardiotoxicity. These compounds, and compositions thereof, may include additional compounds known to be useful for the treatment of cardiac arrhythmias, cardioprotective agents, antibiotics, antiviral agents, or thrombolytic agents (e.g., streptokinase, tissue plasminogen activator, or recombinant tissue plasminogen activator). The compounds and compositions of the invention can have particular usefulness for treating life-threatening ventricular tachyarrhythmias, especially in patients with congestive heart failure (CHF). Post-myocardial infarction patients can also benefit from the administration of the subject compounds and compositions; thus, methods of treating post-myocardial infarction patients are also provided by the subject invention.

Cardioprotective agents include vasodilators and beta blockers described for use in patients with coronary insufficiency (such as those of U.S. Pat. No. 5,175,187 or others known to the skilled artisan). Other cardioprotective agents include known anti-hypertensive agents, e.g., (S)-1-[6-amino-2-[[hydroxy(4phenylbutyl)phosphinyl]oxyl]-L-proline (U.S. Pat. No. 4,962,095) and zofenopril (U.S. Pat. No. 4,931,464). Additional cardioprotective agents include, but are not limited to, aspirin, heparin, warfalin, digitalis, digitoxin, nitroglycerin, isosorbide dinitrate, hydralazine, nitroprusside, captopril, enalapril, and lisinopril. Of particular utility is the combination therapy of the compounds of formula (IA), (IB), and (IC) with carvedilol (Coreg® GlaxoSmithKline) Carvedilol is a nonselective b-adrenergic blocking agent with α₁-blocking activity. It is (±)-1-(Carbazol-4-yloxy)-3-[[2-(o-methoxyphenoxy)ethyl]amino]-2-propanol. When used in combination with caredilol, the compounds of formula (IA), (IB), and (IC), including dexrazoxane, are useful for the treatment of congestive heart failure and sudden death.

The compounds and compositions for formula (IA), (IB), and (IC) also provide effective management for ventricular arrhythmias and supraventricular arrhythmias, including atrial fibrillation and re-entrant tachyarrhythmias involving accessory pathways. Compounds and compositions of the invention are also useful for the treatment of ventricular and supra-ventricular arrhythmias, including atrial fibrillation and flutter, paroxysmal supraventricular tachycardia, ventricular premature beats (VPB), sustained and non-sustained ventricular tachycardia (VT), and ventricular fibrillation (VF). Other non-limiting examples of the arrhythmias which may be treated by the compounds of the instant invention include: narrow QRS tachycardia (atrial, intra-/para-A-V node, or accessory pathway), ventricular tachycardia, and ventricular arrhythmias in cardiomyopathy.

The term “effective amount” is an amount necessary or sufficient to realize a desired biological effect. For example, an effective amount of a compound to treat a condition associated with iron or calcium overload in the amount required to reduce the toxic effects of the condition. For acute iron toxicity, the amount could be the amount necessary to inhibit the associated iron catalyzed free radical generation. For cardiotoxicity, an effective amount is that which is required to reduce or prevent deleterious effects of the cardiotoxin. This may be a protection against elongation of the QT interval. For cardiac arrhythmias, an effective amount may be an amount necessary to resume a regular heart beat, or to positively affect a mechanism associated with irregularity, such as sodium channel blockage, beta blocking or modulating adrenergic receptors, APD modulating, decreasing QT intervals, or calcium channel antagonism. The effective amount may vary, depending, for example, upon the arrhythmic condition treated, weight of the subject and severity of the disease. One of skill in the art can readily determine the effective amount empirically without undue experimentation.

A “subject” or “patient” is meant to describe a human or vertebrate animal including a dog, cat, pocket pet, marmoset, horse, cow, pig, sheep, goat, elephant, giraffe, chicken, monkey, owl, rat, squirrel, slender loris, and mouse.

The compounds of the present invention can be used in the form of salts as in “pharmaceutically acceptable salts” derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, phosphate, sulfate, 3-phenylpropionate, picrate, pivalate, propionate, succinate, tartrate, thiocyanate, p-toluenesulfonate and undecanoate. Also, the basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

The term “metabolite” refers to a compound that is rapidly transformed in vivo from the parent compound, dexrazoxane, for example by hydrolysis in blood. One preferred metabolite of dexrazoxane is ADR-925, which has the following structure:

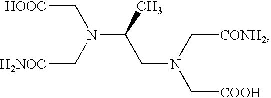

or name: N,N′-[(1S)-1-methyl-1,2-ethanediyl]bis[N-(2-amino-2-oxoethyl)glycine].

Conversely, the term “prodrug” refers to compounds that are rapidly transformed in vivo to yield the parent compound, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987. Prodrugs as described in U.S. Pat. No. 6,284,772 for example may be used as well as those described in Hydrolysis in Drug and Prodrug Metabolism: Chemistry, Biochemistry, and Enzymology, Bernard Testa et al., 2003 and The Organic Chemistry of Drug Design and Drug Action [Chapter 8], Richard Silverman, 2004. Additional prodrugs are described herein, particularly in the Examples section, such as Table 1.

Reference to “dexrazoxane” indicates a compound of the following structure:

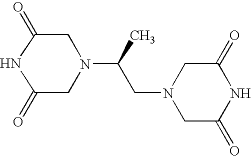

or chemical name: (S)-4,4′-(1-methyl-1,2-ethanediyl)bis-2,6-piperazinedione. The racemate and R-enantiomer, namely (R)-4,4′-(1-methyl-1,2-ethanediyl)bis-2,6piperazinedione are also contemplated to fall within the scope of the present invention. Dexrazoxane is also referred to as ICRF-187 and has associated Trade Names: Cardioxane®, Zinecard®, and Eucardion®.

Reference to “halo,” “halide,” or “halogen” refers to F, Cl, Br, or I atoms, especially F, Cl, and Br.

The phrase “alkyl” refers to substituted and unsubstituted alkyl groups such as methyl, ethyl, propyl, butyl, pentyl, hexyl, heptyl, octyl, nonyl, decyl, undecyl, dodecyl and the like. The phrase “C₁₋₆ alkyl” has the same meaning as alkyl, except that it is limited to alkyl groups of six carbons or less. The phrase C₁₋₆ alkyl also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —CH₂CH(CH₃)₂, —CH₂CH(CH₃)(CH₂CH₃), —CH₂CH(CH₂CH₃)₂, —CH₂C(CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂C(CH₃)₃, —CH(CH₃)CH₂CH(CH₃)₂, —CH(CH₃)CH(CH₃)CH(CH₃), and others. The phrase C₁₋₆ alkyl further includes cyclic alkyl groups such as cyclopropyl, cyclobutyl, cyclopentyl, cyclohexyl, and such rings substituted with straight and branched chain alkyl groups as defined above. The phrase alkyl also includes polycyclic alkyl groups such as, but not limited to, adamantyl norbornyl, and bicyclo[2.2.2]octyl and such rings substituted with straight and branched chain alkyl groups as defined above.

The phrase “aryl” refers to substituted and unsubstituted aryl groups that do not contain heteroatoms. The phrase “C₆₋₁₀ aryl” has the same meaning as aryl, except that it is limited to aryl groups of six to ten carbons atoms. The phrase aryl includes, but is not limited to, groups such as phenyl, biphenyl, and naphthyl by way of example. Aryl groups also include those in which one of the aromatic carbons is bonded to an alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “aryl” includes, but is not limited to tolyl, and hydroxyphenyl among others.

The phrase “alkenyl” refers to straight chain, branched chain, and cyclic groups such as those described with respect to alkyl groups as defined above, except that at least one double bond exists between two carbon atoms. The phrase “C₂₋₆ alkenyl” has the same meaning as alkenyl, except that it is limited to alkenyl groups of two to six carbons. Examples include, but are not limited to, vinyl, —CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, hexadienyl, and the like. The phrase “C₁₋₆ alkenyl” indicates that the double bond may be the point of attachment of the alkenyl group.

The phrase “alkynyl” refers to straight and branched chain groups such as those described with respect to alkyl groups as defined above, except that at least one triple bond exists between two carbon atoms. The phrase “C₂₋₆ alkynyl” has the same meaning as alkynyl, except that it is limited to alkynyl groups of two to six carbons. Examples include, but are not limited to, —C≡C(H),

—C≡C(CH₃), —C≡C(CH₂CH₃), —C(H₂)C≡C(H), —C(H)₂C≡C(CH₃), —C(H)₂C≡C(CH₂CH₃), and the like.

The phrase “alkoxy” refers to groups having the formula —O-alkyl, wherein the point of attachment is the oxy group and the alkyl group is as defined above. The phrase “C₁₋₆ alkoxy” has the same meaning as alkoxy, except that it is limited to alkoxy groups having from one to six carbon atoms.

The phrase “aryloxy” refers to groups having the formula —O-aryl, wherein the point of attachment is the oxy group and the aryl group is as defined above. The phrase “C₆₋₁₀ aryloxy” has the same meaning as aryloxy, except that it is limited to aryloxy groups of six to ten carbon atoms.

The phrase “heterocyclyl” refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridyl, dihydropyridyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl, 2H-tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms such as, but not limited to furanyl; unsaturated 3 to 8 membered rings containing 1 to 2 oxygen atoms and 1 to 3 nitrogen atoms such as, but not limited to, oxazolyl, isoxazolyl, oxadiazolyl (e.g. 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4dihydrobenzothiazinyl, etc.), unsaturated condensed heterocyclic rings containing 1 to 2 oxygen atoms such as benzodioxolyl (e.g. 1,3-benzodioxoyl, etc.); unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene, tetrahydrothiophene oxide, and tetrahydrothiophene 1,1dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4-triazole, tetrazole, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran. “Heterocyclyl” also refers to those groups as defined above in which one of the ring members is bonded to a non-hydrogen atom such as described above with respect to substituted alkyl groups and substituted aryl groups. Examples, include, but are not limited to, 2methylbenzimidazolyl, 5-methylbenzimidazolyl, 5-chlorobenzthiazolyl, 1-methyl piperazinyl, and 2-chloropyridyl among others. Heterocyclyl groups are those limited to having 2 to 15 carbon atoms and as many as 6 additional heteroatoms as described above. More preferred heterocyclyl groups have from 3 to 5 carbon atoms and as many as 2 heteroatoms. Most preferred heterocyclyl groups include piperidinyl, pyrrolidinyl, azetidinyl, and aziridinyl groups.

The term “substituted” refers to the replacement of one or more hydrogen atom with a monovalent or divalent radical. Suitable substitution groups include, for example, hydroxyl, nitro, amino, imino, cyano, halo, thio, thioamido, amidino, imidino, oxo, oxamidino, methoxamidino, imidino, guanidino, sulfonamido, carboxyl, formyl, alkyl, heterocyclyl, aryl, haloalkyl, alkoxy, alkoxyalkyl, alkylcarbonyl, arylcarbonyl, aralkylcarbonyl, alkylthio, aminoalkyl, alkylamino, cyanoalkyl, and the like. For example, one preferred “substituted C₁₋₆ alkyl” is tertbutanol. Another preferred substituted C₁₋₆ alkyl is —CH₂C(CH₃)₂NH—SO₂CH₃.

The substitution group can itself be substituted one time. For example, an alkoxy substituent of an alkyl group may be substituted with a halogen, and oxo group, an aryl group, or the like. The group substituted onto the substitution group can be carboxyl, halo, nitro, oxo, amino, cyano, hydroxyl, C₁₋₆ alkyl, C₁₋₆ alkoxy, C₆₋₁₀ aryl, aminocarbonyl, —SR, thioamido, —SO₃H, —SO₂R or cycloalkyl, where R is typically hydrogen, hydroxyl or C₁₋₆ alkyl.

When the substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon atoms or heteroatoms.

Dexrazoxane, metabolites, or derivatives thereof of formula I may exhibit the phenomenon of tautomerism, and the formula drawings within this specification can represent only one of the possible tautomeric forms. It is to be understood that the invention encompasses any tautomeric form which possesses antiarrhythmic activity and is not to be limited merely to any one tautomeric form utilized within the formula drawings.

Dexrazoxane, metabolites, or derivatives thereof of formula I also may exist in solvated as well as unsolvated forms such as, for example, hydrated forms. The invention encompasses both solvated and unsolvated forms which possess activity.

The compounds of this invention can be administered as the sole active ingredient or in combination with other antiarrhythmic agents or other cardiovascular agents. The compounds, or pharmaceutically acceptable salts thereof, of the present invention, in the described dosages, are administered orally, intraperitoneally, subcutaneously, intramuscularly, transdermally, sublingually or intravenously. They are preferably administered intravenously, for example reconstituted in an aqueous solution. The amount of active compound in such therapeutically useful compositions or preparations is such that a suitable dosage will be obtained.

Commercially, dexrazoxane is currently formulated as a lyophilized acid salt of hydrochloride acid. The acid salt is unstable in aqueous solution and so is lyophilized for stability. To manufacture the commercial product, dexrazoxane is dissolved in 0.1 M HCl at a concentration of 20 mg/ml and placed into vials. The vials are partially stoppered and loaded on the shelves of a lyophilization chamber. Compounding and vial-filling processes are carried out at ambient temperature. The solutions are frozen and the chamber evacuated until lyophilization is complete.

Typically, the solution occupies less than the full vial volume to optimize lyophilization time and to allow for expansion of the solution upon freezing. The vials are then supplied to clinics in 250 mg or 500 mg doses. When needed, dexrazoxane is reconstituted in the clinic. A current recommended dose is 2 g for an adult human. Thus, preparing a dose for injection previously required pooling four 500 mg doses.

Dexrazoxane is subject to decomposition in aqueous solutions. Because of this, the time span from compounding to beginning of freezing in the lyophilizer must not exceed 7 hours without significant decomposition. Lyophilized dexrazoxane is stable for extended periods of time, on the order of 12 months without significant decomposition. Reconstituted dexrazoxane is stable for about 6 hours at room temperature.

The solubility of dexrazoxane in water at 25° C. is about 10 mg/ml water. Dexrazoxane (ICRF-187) and its racemate, razoxane (ICRF-159) are sparingly soluble in 0.1 N HCl, slightly soluble in polar solvents such as ethanol and methanol and practically insoluble in non-polar, organic solvents [Repta, Baltezor and Bansal, J. Pharm Sci. 65 (1976) 238-242]. Generally speaking, the solubility of dexrazoxane increases as pH decreases. Formulating dexrazoxane as a salt of hydrochloric acid improves its solubility. Solubility of hydrochloric acid as high as 35 mg/ml in 0.1 N HCl at 25° C. has been reported (U.S. Pat. No. 4,963,551), though therapeutic concentrations around 20-25 mg/ml are preferred (see U.S. Pat. No. 5,760,039, hereby incorporated by reference). A 20 mg/ml solution is known to be used commercially. The concentration of dexrazoxane in the bulk solutions may also be 0.01 N HCl is a 0.13 M dexrazoxane (the molecular weight of dexrazoxane being 268.28 g/mol).

Therapeutic and prophylactic application of the subject compounds, and compositions comprising them, can be accomplished by any suitable method and technique presently or prospectively known to those skilled in the art. Further, the compounds of the invention have use as starting materials or intermediates for the preparation of other useful compounds and compositions.

The compounds of the invention are useful for various non-therapeutic and therapeutic purposes.

The administration of the subject compounds of the invention is useful as an antiarrhythmic agent. Thus, pharmaceutical compositions containing compounds of the invention as active ingredients are useful in prophylactic or therapeutic treatment of cardiac arrhythmias, conditions associated with iron and calcium overload, environmentally induced and pharmaceutically induced cardiotoxicity, and, in particular, cancer chemotherapy induced cardiotoxicity in humans or other mammals.

The dosage administered will be dependent upon the response desired; the type of host involved; its age, health, weight, kind of concurrent treatment, if any; frequency of treatment; therapeutic ratio and like considerations. Expressed in terms of concentration, dexrazoxane can be present in the new compositions for use dermally, intranasally, bronchially, intramuscularly, intravaginally, intravenously, or orally in a concentration of from about 0.01 to about 50% w/w of the composition, and especially from about 0.1 to about 30% w/w of the composition. Preferably, the compound is present in a composition from about 1 to about 10% and, most preferably, the novel composition comprises about 5% compound.

The compositions of the invention are advantageously used in a variety of forms, e.g., tablets, ointments, capsules, pills, powders, aerosols, granules, and oral solutions or suspensions and the like containing the indicated suitable quantities of the active ingredient. Such compositions are referred to herein and in the accompanying claims generically as “pharmaceutical compositions.” Typically, they can be in unit dosage form, namely, in physically discrete units suitable as unitary dosages for human or animal subjects, each unit containing a predetermined quantity of active ingredient calculated to produce the desired therapeutic or prophylactic effect in association with one or more pharmaceutically acceptable other ingredients, e.g., diluent or carrier.

Where the pharmaceutical compositions are aerosols, the active ingredients can be packaged in pressurized aerosol containers with a propellant, e.g., carbon dioxide, nitrogen, propane, etc. with the usual adjuvants such as cosolvents, wetting agents, etc.

Where the pharmaceutical compositions are ointments, the active ingredient can be mixed with a diluent vehicle such as cocoa butter, viscous polyethylene glycols, hydrogenated oils, and such mixtures can be emulsified if desired.

In accordance with the invention, pharmaceutical compositions comprise, as an inactive ingredient, an effective amount of one or more non-toxic, pharmaceutically acceptable ingredient(s). Examples of such ingredients for use in the compositions include ethanol, dimethyl sulfoxide, glycerol, silica, alumina, starch, calcium carbonate, talc, flour, and equivalent non-toxic carriers and diluents. Additionally, adjuvants may be administered in conjunction with the compositions described herein for treatment of sudden death, cardiac toxicity and calcyclin and other calcium binding protein mediated diseases.

### Compositions and Methods for Inhalation

In an embodiment of the invention, dry powder and liquid aerosol formulations and methods for their administration via inhalation are provided. Inhalation into the lung is an important route of administration. Once delivered to the lung, the compound is absorbed into the pulmonary veins and delivered directly into the left atrium of the heart. Delivered to the heart in this manner, the dexrazoxane or non-dexrazoxane compound of the formula (IA), (IB), or (IC) is presented directly at the desired site of action to exert a desired cardiac effect. The desired effect can be cardioprotective, antiarrhythmic, anti-QT prolongation, or other additional cardiac effects as described in the present application. By delivering the drug agent directly to the heart, the required efficacious dose is generally lower. Delivery of dexrazoxane or a non-dexrazoxane compound of the formula (IA), (IB), or (IC) to the heart via inhalation also results in reduced systemic exposure and lowered toxicity or lowered incident of adverse effects.

### Dry Powder Formulations

In the practice of the present invention, the aerosol powder is inhaled by the human or animal subject, and thereby enters the lungs of the human or animal subject. The aerosol powder comprises particles that comprise the dexrazoxane or a non-dexrazoxane compound of the formula (IA), (IB), or (IC). For the purposes of the discussion of liquid and dry aerosol formulations, the term “cardioprotectant” will be defined meaning a dexrazoxane or a non-dexrazoxane compound of the formula (IA), (IB), or (IC). It has been found that aerosol powders (comprising a cardioprotectant) wherein at least 50% of the particles have an aerodynamic diameter in the range of from 1 μm to 5 μm effectively penetrate into the lungs of the human or animal subject, thereby effectively delivering the dexrazoxane or a non-dexrazoxane compound of the formula (IA), (IB), or (IC) to the lungs of the subject. By way of example, some aerosol powders (comprising a cardioprotectant) useful in the practice of the present invention comprise particles wherein at least 60% of the particles, or at least 70% of the particles, or at least 80% of the particles, or at least 90% of the particles, or at least 95% of the particles, have an aerodynamic diameter in the range of from 1 μm to 5 μm.

The term “aerodynamic diameter” refers to the diameter of a unit-density sphere having the same terminal settling velocity as the particle in question (see, e.g., “Aerosol Measurement: Principles, Techniques and Applications”. Edited by Klaus Willeke and Paul A. Baron. Van Nostrand Reinhold, New York, 1993). Aerodynamic diameter is used, for example, to predict where such particles will be deposited in the respiratory tract.

“Mass median aerodynamic diameter” (abbreviated as MMAD) is a measure of the aerodynamic size of a dispersed particle. The aerodynamic size distribution defines the manner in which an aerosol deposits during inhalation, and is the diameter of a unit density sphere having the same settling velocity, generally in air, as the particle. The aerodynamic diameter encompasses particle shape, density and physical size of a particle. When there is a log-normal distribution, the aerodynamic size distribution may be characterized by the mass median aerodynamic diameter (MMAD). As used herein, MMAD refers to the midpoint or median of the aerodynamic particle size distribution of an aerosolized powder determined by Anderson cascade impaction.

In brief, cascade impaction devices include a series of screens of decreasing pore size. The screens trap particles within a moving jet that passes through the impactor. The amount of particulate material (having particle sizes within a defined size range) that is trapped on each screen can be determined by washing the screen and measuring the amount of eluted material. Examples of cascade impactors, and their use, are described in Chapter 601 (Aerosols) of the Pharmacopoeia of the United States (26th Revision), the cited portion of which publication is incorporated herein by reference.

The powdered cardioprotectant formulations useful in the practice of the present invention typically contain less than 15% by weight moisture, usually below about 1% by weight, and preferably below about 8% by weight.

In the practice of the present invention a therapeutically effective amount of an aerosol powder comprising a dexrazoxane or a non-dexrazoxane compound of the formula (IA), (IB), or (IC) is administered by inhalation to a patient suffering from cardiotoxicity, arrhythmia, QT prolongation, or other cardiac condition. A therapeutically effective amount of an aerosol powder contains sufficient dexrazoxane or a non-dexrazoxane compound of the formula (IA), (IB), or (IC) to completely or partially prevent cardiotoxicity from exposure to a cardiotoxin, reverse or prevent QT prolongation or arrhythmia, prevent sudden death, Torsade de pointes, or another cardiac condition or syndrome. As a representative example, for cardioprotectants, therapeutically effective amounts are obtained by administering to a patient from once daily to five times a day, and in preferred aspects of the invention once or twice a day, an aerosol powder compositions comprising a dosage from about 25 mg to about 500 mg, more preferably from about 50 mg to about 300 mg, and most preferably from about 50 mg to about 200 mg of dexrazoxane or a non-dexrazoxane compound of the formula (IA), (IB), or (IC) (determined as free-base weight excluding the weight of counterion(s) that may be present).

The dosage of administered cardioprotectant may be administered from a single container as a single unit dose, or it may be divided into multiple containers or units doses for sequential administration, depending of the inhalation device used for delivery of the compound. The dry powder aerosol compositions of the invention are administered to a patient for a single treatment or for multiple treatments over a course of two or more days to several weeks. The treatments may also be continued on as maintenance treatments.

The aerosol powder typically comprises from 20% (by weight) to 90% (by weight) of dexrazoxane or a non-dexrazoxane compound of the formula (IA), (IB), or (IC). Thus, in some embodiments of the present invention the aerosol powder comprises from 30% (by weight) to 80% (by weight) of a dexrazoxane or a non-dexrazoxane compound of the formula (IA), (IB), or (IC). In some embodiments of the present invention the aerosol powder comprises from 40% (by weight) to 70% (by weight) of a dexrazoxane or a non-dexrazoxane compound of the formula (IA), (IB), or (IC). In this context, the percentage (by weight) of the dexrazoxane or a non-dexrazoxane compound of the formula (IA), (IB), or (IC) refers to the amount of the free compound, excluding the weight of counterion(s) that may be present.

Aerosol powders of the invention typically, but not necessarily, include at least one physiologically acceptable carrier. For example, the aerosol powder can include one or more excipients, and/or any other component that improves the effectiveness of the dexrazoxane or a non-dexrazoxane compound of the formula (IA), (IB), or (IC). Such excipients may serve simply as bulking agents when it is desired to reduce the active agent concentration in the powder which is being delivered to a patient. Such excipients may also serve to improve the dispersability of the powder within a powder dispersion device in order to provide more efficient and reproducible delivery of the active agent and to improve the handling characteristics of the active agent (e.g., flowability and consistency) to facilitate manufacturing and powder filling. In particular, the excipient materials can often function to improve the physical and chemical stability of the cardioprotectant, to minimize the residual moisture content and hinder moisture uptake, and to enhance particle size, degree of aggregation, surface properties (e.g., rugosity), ease of inhalation, and targeting of the resultant particles to the deep lung.

Pharmaceutical excipients and additives useful in the cardioprotectant compositions useful in the practice of the present invention include, but are not limited to, proteins, peptides, amino acids, lipids, polymers, and carbohydrates (e.g., sugars, including monosaccharides, di-, tri-, tetra-, and oligosaccharides; derivatized sugars such as alditols, aldonic acids, esterified sugars; and polysaccharides or sugar polymers), which may be present singly or in combination. Exemplary protein excipients include serum albumin such as human serum albumin (HSA), recombinant human albumin (rHA), gelatin, and casein. Representative amino acid/polypeptide components, which may also function in a buffering capacity, include alanine, glycine, arginine, betaine, histidine, glutamic acid, aspartic acid, cysteine, lysine, leucine, proline, isoleucine, valine, methionine, phenylalanine, and aspartame. Polyamino acids of the representative amino acids such as di-leucine and tri-leucine are also suitable for use with the present invention.

Carbohydrate excipients suitable for use in the invention include, for example, monosaccharides such as fructose, maltose, galactose, glucose, D-mannose, and sorbose; disaccharides, such as lactose, sucrose, trehalose, cellobiose; polysaccharides, such as raffinose, melezitose, maltodextrins, dextrans, and starches; and alditols, such as mannitol, xylitol, maltitol, lactitol, xylitol sorbitol (glucitol), and myoinositol.

The cardioprotectant compositions may also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers.

Additionally, the cardioprotectant compositions useful in the practice of the invention may include polymeric excipients/additives such as polyvinylpyrrolidones, hydroxypropyl methylcellulose, methylcellulose, ethylcellulose, Ficolls (a polymeric sugar), dextran, dextrates (e.g., cyclodextrins, such as 2-hydroxypropyl-β-cyclodextrin, hydroxyethyl starch), polyethylene glycols, pectin, salts (e.g., sodium chloride), antioxidants, antistatic agents, surfactants (e.g., polysorbates such as “TWEEN 20” and “TWEEN 80”, lecithin, oleic acid, benzalkonium chloride, and sorbitan esters), lipids (e.g., phospholipids, fatty acids), steroids (e.g., cholesterol), and chelating agents (e.g., EDTA). Other examples of pharmaceutical excipients and/or additives suitable for use in the cardioprotectant compositions are listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are herein incorporated by reference.

The cardioprotectant compositions useful in the practice of the invention may include a dispersing agent for improving the intrinsic dispersability properties of the cardioprotectant powders. Suitable agents are disclosed in PCT applications WO 95/31479, WO 96/32096, and WO 96/32149, hereby incorporated in their entirety by reference. As described therein, suitable agents include water soluble polypeptides and hydrophobic amino acids such as tryptophan, leucine, phenylalanine, and glycine. Leucine and tri-leucine are particularly preferred for use according to this invention.

The solid state matrix formed by the cardioprotectant and excipient imparts a stabilizing environment to the cardioprotectant. The stabilizing matrix may be crystalline, an amorphous glass, or a mixture of both forms. Most suitable are dry powder formulations which are a mixture of both forms. For cardioprotectant dry powder formulations which are substantially amorphous, preferred are those formulations exhibiting glass transition temperatures (Tg) above about 35° C., preferably above about 45° C., and more preferably above about 55° C. Preferably, Tg is at least 20° C. above the storage temperature. According to a preferred embodiment, the cardioprotectant compositions comprise a phospholipid as the solid state matrix as disclosed in WO 99/16419 and WO 01/85136, hereby incorporated in their entirety by reference.

Dry powder cardioprotectant compositions may be prepared by spray drying under conditions which result in a substantially amorphous glassy or a substantially crystalline bioactive powder as described above. Spray drying of the cardioprotectant-solution formulations is carried out, for example, as described generally in the “Spray Drying Handbook,” 5th ed., K. Masters, John Wiley & Sons, Inc., NY, N.Y. (1991), and in WO 97/41833.

To prepare a cardioprotectant solution for spray-drying according to one embodiment of the invention, a cardioprotectant is generally dissolved in a physiologically acceptable solvent such as water. The pH range of solutions to be spray-dried is generally maintained between about 3 and 10, preferably 5 to 8, with near neutral pHs being preferred, since such pHs may aid in maintaining the physiological compatibility of the powder after dissolution of powder within the lung. The aqueous formulation may optionally contain additional water-miscible solvents, such as alcohols, acetone, and the like. Representative alcohols are lower alcohols such as methanol, ethanol, propanol, isopropanol, and the like. Cardioprotectant solutions will generally contain cardioprotectant dissolved at a concentration from 0.05% (weight/volume) to about 35% (weight/volume), usually from 0.5% to 10.0% (weight/volume).

The cardioprotectant-containing solutions are then spray dried in a conventional spray drier, such as those available from commercial suppliers such as Niro A/S (Denmark), Buchi (Switzerland), and the like, resulting in a stable, cardioprotectant dry powder. Optimal conditions for spray drying the cardioprotectant solutions will vary depending upon the formulation components, and are generally determined experimentally. The gas used to spray dry the material is typically air, although inert gases such as nitrogen or argon are also suitable. Moreover, the temperature of both the inlet and outlet of the gas used to dry the sprayed material is such that it does not cause deactivation of cardioprotectant in the sprayed material. Such temperatures are typically determined experimentally, although generally, the inlet temperature will range from about 50° C. to about 200° C. while the outlet temperature will range from about 30° C. to about 150° C.

Cardioprotectant dry powders may also be prepared by lyophilization, vacuum drying, spray freeze drying, super critical fluid processing, or other forms of evaporative drying or by blending, grinding, or jet milling formulation components in dry powder form. In some instances, it may be desirable to provide the cardioprotectant dry powder formulation in a form that possesses improved handling/processing characteristics, e.g., reduced static, better flowability, low caking, and the like, by preparing compositions composed of fine particle aggregates, that is, aggregates or agglomerates of the above-described cardioprotectant dry powder particles, where the aggregates are readily broken back down to the fine powder components for pulmonary delivery, as described, e.g., in U.S. Pat. No. 5,654,007. Alternatively, the cardioprotectant powders may be prepared by agglomerating the powder components, sieving the materials to obtain the agglomerates, spheronizing to provide a more spherical agglomerate, and sizing to obtain a uniformly-sized product, as described, e.g., in WO 95/09616. The cardioprotectant dry powders are preferably maintained under dry (i.e., relatively low humidity) conditions during manufacture, processing, and storage.

According to one embodiment, an exemplary powdered dexrazoxane or a non-dexrazoxane compound of the formula (IA), (IB), or (IC) formulation useful in the practice of the present invention may be made according to the emulsification/spray drying process disclosed in WO 99/16419 and WO 01/85136 cited above. Formulations according to such embodiments are engineered to comprise dry powder particles comprising at least 75% w/w dexrazoxane or a non-dexrazoxane compound of the formula (IA), (IB), or (IC), preferably at least 85% w/w dexrazoxane or a non-dexrazoxane compound of the formula (IA), (IB), or (IC), 2-25% w/w of a phospholipid, preferably 8-18% w/w, and 0-5% w/w of a metal ion such as calcium chloride. The particles of this embodiment generally have an MMAD of from 1 micron to 5 microns, and a bulk density of greater than 0.08 g/cm³, preferably greater than 0.12 g/cm³.

Another exemplary powdered dexrazoxane or a non-dexrazoxane compound of the formula (IA), (IB), or (IC) formulation useful in the practice of the present invention may be produced by creating an emulsion containing active dexrazoxane or a non-dexrazoxane compound of the formula (IA), (IB), or (IC), 1,2-distearoyl-sn-glycero-3-phosphocholine (DSPC), CaCl2 and perfluorooctyl bromide (PFOB). This feedstock emulsion is then sprayed through an atomizer nozzle, producing fine droplets. As the droplets dry, water and PFOB evaporate yielding phospholipid-based spherical particles with porous structure. These spheres are of low density and thus demonstrate favorable aerodynamic characteristics (e.g., the spherical particles have an aerodynamic diameter in the range of from 1 μm to 5 μm). Their high surface porosity also reduces particle-to-particle contact, decreasing the energy required for aerosol suspension.

The aerosol powder (comprising a dexrazoxane or a non-dexrazoxane compound of the formula (IA), (IB), or (IC)) can be administered using a dry powder inhaler that uses a patient's (e.g., human's or animal's) inhaled breath to deliver the powdered dexrazoxane or a non-dexrazoxane compound of the formula (IA), (IB), or (IC) formulation to the lungs. An example of a useful dry powder inhaler is the model T-326 inhaler sold by Nektar Therapeutics, 150 Industrial Road, San Carlos, Calif. 94070, U.S.A. Other examples of useful dry powder inhalation devices are described in U.S. Pat. Nos. 5,458,135; 5,740,794; 5,775,320; and 5,785,049. When administered using a device of this type, the powdered medicament is contained in a receptacle having a puncturable lid or other access surface, preferably a blister package or cartridge, where the receptacle may contain a single dosage unit or multiple dosage units. Exemplary methods for filling large numbers of cavities with metered doses of dry powder medicament are described in U.S. Pat. No. 5,826,633.

Also suitable for delivering the cardioprotectant powders described herein are dry powder inhalers of the type described, for example, in U.S. Pat. Nos. 3,906,950, 4,013,075, 4,069,819, and 4,995,385, wherein a premeasured dose of cardioprotectant dry powder for delivery to a subject is contained within a capsule, such as a hard gelatin capsule. The size of the capsule, such as 00, 0, No. 1, or No. 3 sized capsules, depends, among other factors, upon the inhalation device used to administer the powders.

Other dry powder dispersion devices for pulmonarily administering cardioprotectant dry powders include those described, for example, in EP 129985, EP 472598, EP 467172, and U.S. Pat. No. 5,522,385. Also suitable for delivering the cardioprotectant dry powders of the invention are inhalation devices such as the Astra-Draco “TURBUHALER”. This type of device is described in detail in U.S. Pat. Nos. 4,668,218, 4,667,668, and 4,805,811.

Also suitable are devices which employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device, such as described in U.S. Pat. No. 5,388,572.

In view of the foregoing, it will be understood that a therapeutically effective amount of an aerosol powder (comprising an dexrazoxane or a non-dexrazoxane compound of the formula (IA), (IB), or (IC)) can be administered from a single container, or from more than one container, disposed within a dry powder inhalation device. For example, a dry powder inhalation device may be loaded with a single container containing a therapeutically effective amount of an aerosol powder (comprising a dexrazoxane or a non-dexrazoxane compound of the formula (IA), (IB), or (IC)), and the contents of the container are inhaled by a human or animal subject. Again by way of example, a dry powder inhaler may be loaded with multiple unit dose containers (e.g., 2, 3, or 4 containers), such as #2 HPMC capsules, that separately contain less than a therapeutically effective amount of an aerosol powder (comprising an dexrazoxane or a non-dexrazoxane compound of the formula (IA), (IB), or (IC)), but which together contain a therapeutically effective amount of the aerosol powder. The dry powder inhaler discharges the contents of all of the containers disposed therein, and thereby provides the user with a therapeutically effective amount of the aerosol powder.

The emitted dose (ED) of the powdered dexrazoxane or a non-dexrazoxane compound of the formula (IA), (IB), or (IC) formulations is greater than 50%. More preferably, the ED of the powdered dexrazoxane or a non-dexrazoxane compound of the formula (IA), (IB), or (IC) formulations useful in the practice of the present invention is greater than 70%, and is often greater than 80%. As used herein, the term “emitted dose” or “ED” refers to an indication of the delivery of dry powder from a suitable inhaler device after a firing or dispersion event from a powder unit, capsule, or reservoir. ED is defined as the ratio of the dose delivered by an inhaler device to the nominal dose (i.e., the mass of powder per unit dose placed into a suitable inhaler device prior to firing). The ED is an experimentally-determined amount, and is typically determined using an in-vitro device set up which mimics patient dosing. To determine an ED value, a nominal dose of dry powder (as defined above) is placed into a suitable dry powder inhaler, which is then actuated, dispersing the powder. The resulting aerosol cloud is then drawn by vacuum from the device, where it is captured on a tared filter attached to the device mouthpiece. The amount of powder that reaches the filter constitutes the delivered dose. For example, for a 50 mg, dry powder-containing #2 capsule placed into an inhalation device, if dispersion of the powder results in the recovery of 40 mg of powder on a tared filter as described above, then the ED for the dry powder composition is: 40 mg (delivered dose)/50 mg (nominal dose)×100=80%.

### Liquid Aerosols

Aerosolization of cardioprotectants utilize delivery of aerosolized dexrazoxane or a non-dexrazoxane compound of the formula (IA), (IB), or (IC) or a pharmaceutically acceptable salt thereof, or a mixture of salts using atomizing, jet, ultrasonic; electronic or other equivalent nebulizers. Those which are portable, such as atomizing, ultrasonic and electronic nebulizers are preferred for ambulatory treatment. The jet nebulizers with a compressor nebulize the dexrazoxane or a non-dexrazoxane compound of the formula (IA), (IB), or (IC) formulation very efficiently but are more suitable for use in the hospital and doctor's office.

The dosing regimen for the liquid aerosol formulations of dexrazoxane or a non-dexrazoxane compound of the formula (IA), (IB), or (IC) comprises from one to four, typically, or more than four times daily, in atypical cases, administration of the aerosol.

A primary requirement of this invention is to deliver dexrazoxane or a non-dexrazoxane compound of the formula (IA), (IB), or (IC) efficiently to the endobronchial space of airways. Thus, the invention requires that at least 30-50%, preferably 70-90% of the active drug, that is dexrazoxane or a non-dexrazoxane compound of the formula (IA), (IB), or (IC) or a salt thereof, subjected to nebulization is in fact delivered to a site where it is efficiently transported to its site of action.

The compositions of the invention described herein provide the drug formulated in a solution permitting delivery of a therapeutically efficient amount of the drug, provided that the aerosol generated by the nebulization meets criteria required for such efficient delivery. There are quite a few nebulizer types currently commercially available. A nebulizer is selected primarily on the basis of allowing the formation of a cardioprotectant aerosol having a mass medium average diameter predominantly between 1 to 5 μm. The delivered amount of dexrazoxane or a non-dexrazoxane compound of the formula (IA), (IB), or (IC) must be efficacious for treatment and prophylaxis of cardiotoxicity, arrhythmia, QT prolongation, or other cardiac condition. The selected nebulizer thus must be able to efficiently aerosolize the formulation which has salinity, osmotic strength, and pH adjusted as to permit generation of a cardioprotectant aerosol that is therapeutically effective and well tolerated by patients. The nebulizer must be able to handle the formulation having a smallest possible aerosolizable volume and still able to deliver effective dose of dexrazoxane or a non-dexrazoxane compound of the formula (IA), (IB), or (IC) to lungs for effective transport to, and distribution into, the heart. Additionally, the aerosolized formulation must not impair the functionality of the airways and must minimize undesirable side effects.

In order to be therapeutically effective, the majority of aerosolized dexrazoxane or a non-dexrazoxane compound of the formula (IA), (IB), or (IC) particles should not have larger mass medium average diameter (MMAD) than between about 1 to 5 μm. When the aerosol contains a large number of particles with a MMAD larger than 5 μm, these are deposited in the upper airways decreasing the amount of cardioprotectant delivered to the lungs and, ultimately, the heart. Nebulizers suitable for practicing this invention are typically able to nebulize large and small volumes of the cardioprotectant formulations efficiently, that is into the aerosol particle size predominantly in the range from about 1 to 5 μm. In this context, this means that at least 70% but preferably more than 90% of all generated aerosol particles are within about 1 to 5 μm range.

Jet and ultrasonic nebulizers can produce and deliver particles between the 1 and 5 μm particle size. A jet nebulizer utilizes air pressure breakage of an aqueous dexrazoxane or a non-dexrazoxane compound of the formula (IA), (IB), or (IC) solution into aerosol droplets. An ultrasonic nebulizer utilizes shearing of the aqueous dexrazoxane or a non-dexrazoxane compound of the formula (IA), (IB), or (IC) solution by a piezoelectric crystal. One type of nebulizer which is suitable and preferred for dexrazoxane or a non-dexrazoxane compound of the formula (IA), (IB), or (IC) delivery is an atomizing nebulizer which consists of a liquid storage container in fluid contact with the diaphragm and inhalation and exhalation valves. For administration of the dexrazoxane or a non-dexrazoxane compound of the formula (IA), (IB), or (IC) formulation, 1 to 30 ml of the formulation is placed in the storage container, aerosol generator is engaged which produces atomized aerosol of particle sizes selectively between about 1 to 5 μm. Typical nebulizing devices suitable for practicing this invention include atomizing nebulizers, or modified jet nebulizers, ultrasonic nebulizers, electronic nebulizers, and vibrating porous plate nebulizers for handling concentrated cardioprotectant solutions in a specific formulation having a specific pH, osmolality and salinity. An example of an electronic nebulizer is the PARI inhalation nebulizer described in PCT/US00/29541. A jet nebulizer utilizes air pressure to break a liquid solution into aerosol droplets. An ultrasonic nebulizer works by a piezoelectric crystal that shears a liquid into small aerosol droplets. A pressurized nebulization system forces solution under pressure through small pores to generate aerosol droplets. A vibrating porous plate device utilizes rapid vibration to shear a stream of liquid into appropriate droplet sizes.

In the practice of the present invention, a therapeutically effective amount of a liquid aerosol comprising a dexrazoxane or a non-dexrazoxane compound of the formula (IA), (IB), or (IC) is administered by inhalation to a patient suffering from cardiotoxicity, arrhythmia, QT prolongation, other cardiac condition, or a condition associated with iron or calcium overload. A therapeutically effective amount of a liquid aerosol contains sufficient dexrazoxane or a non-dexrazoxane compound of the formula (IA), (IB), or (IC) to completely or partially prevent cardiotoxicity from exposure to a cardiotoxin, reverse or prevent QT prolongation or arrhythmia, prevent sudden death, Torsade de pointes, or another cardiac condition or syndrome. As a representative example, for cardioprotectants, therapeutically effective amounts are obtained by administering to a patient from once daily to five times a day, and in preferred aspects of the invention once or twice a day, a liquid aerosol composition comprising a dosage from about 40 mg to about 1000 mg, more preferably from about 50 mg to about 1000 mg, and most preferably from about 75 mg to about 800 mg of dexrazoxane or a non-dexrazoxane compound of the formula (IA), (IB), or (IC) (determined as free-base weight excluding the weight of counterion(s) that may be present). In a preferred embodiment, dexrazoxane is administered to a patient in need of cardioprotection. In particular, the patient may be undergoing cancer chemotherapy. More particularly, the patient is undergoing cancer chemotherapy with an anthracycline chemotherapeutic agent. In a further embodiment, the agent is daunorubicin, doxorubicin, epirubicin, or idarubicin. In an additional embodiment, the patient is undergoing cancer chemotherapy with an EGFR modulating agent. In a further embodiment, the agent is trastuzumab (Herceptin), cetuximab (Erbitux), bevacizumab (Avastin), panitumumab, gefitinib (Iressa), or erlotinib (Tarceva). In another embodiment, the patient is undergoing combination cancer therapy with an EGFR modulating agent and an anthracycline chemotherapeutic agent. In a method of the invention, the cardioprotective treatment with dexrazoxane is at a reduced dose than is required from treatment by an IV dose. In an aspect of the embodiment, the dose is about 0.75× or less than that which is the necessary IV dose of dexrazoxane. In a further aspect of the embodiment, the dose is about 0.5× or less than that which is the necessary IV dose of dexrazoxane. In a further aspect of the embodiment, the dose is about 0.25× or less than that which is the necessary IV dose of dexrazoxane.

In an alternate embodiment, a non-dexrazoxane compound of formula (IA), (IB), or (IC) is administered to a patient in need of cardioprotection. In particular, the patient may be undergoing cancer chemotherapy. More particularly, the patient is undergoing cancer chemotherapy with an anthracycline chemotherapeutic agent. In a further embodiment, the agent is daunorubicin, doxorubicin, epirubicin, or idarubicin. In an additional embodiment, the patient is undergoing cancer chemotherapy with an EGFR modulating agent. In a further embodiment, the agent is trastuzumab (Herceptin), cetuximab (Erbitux), bevacizumab (Avastin), panitumumab, gefitinib (Iressa), or erlotinib (Tarceva). In another embodiment, the patient is undergoing combination cancer therapy with an EGFR modulating agent and an anthracycline chemotherapeutic agent. In a method of the invention, the cardioprotective treatment with dexrazoxane is at a reduced dose than is required from treatment by an IV dose. In an aspect of the embodiment, the dose is about 0.75× or less than that which is the necessary IV dose of dexrazoxane. In a further aspect of the embodiment, the dose is about 0.5× or less than that which is the necessary IV dose of dexrazoxane. In a further aspect of the embodiment, the dose is about 0.25× or less than that which is the necessary IV dose of dexrazoxane.

The dosage of administered cardioprotectant may be administered from a single container as a single unit dose, or it may be divided into multiple containers or units doses for sequential administration, depending of the inhalation device used for delivery of the compound. The liquid aerosol compositions of the invention are administered to a patient for a single treatment or for multiple treatments over a course of two or more days to several weeks. The treatments may also be continued on as maintenance treatments.

The cardioprotectant for use in the current invention is typically prepared for liquid aerosols in the form of salts derived from inorganic or organic acids. These salts include but are not limited to the following salts: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, persulfate, 3-phenylpropionate, picrate, pivalate, propionate, succinate, tartrate, thiocyanate, p-toluenesulfonate and undecanoate.

Examples of acids which may be employed to form pharmaceutically acceptable acid addition salts for liquid aerosols include such inorganic acids as hydrochloric acid, sulphuric acid and phosphoric acid and such organic acids as, for example, oxalic acid, maleic acid, acetic, aspartic, succinic acid and citric acid. Basic addition salts can be prepared in situ during the final isolation and purification of a compound or separately by reacting the carboxylic or sulfuric acid function with a suitable base such as the hydroxide, carbonate or bicarbonate of a pharmaceutically acceptable metal cation or with ammonia, or with an organic primary, secondary or tertiary amine. Pharmaceutical acceptable salts for liquid aerosols also include, but are not limited to, cations based on the alkali and alkaline earth metals, such as sodium, lithium, potassium, calcium, magnesium or aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, amino acids including basic amino acids (i.e. lysine, histidine, ornithine) and the like. Other representative organic amines useful for the formation of base addition salts include diethylamine, ethylenediamine, ethanolamine, diethylamine and the like.

These compositions ideally will be formulated into a liquid (solution, suspension, emulsion etc.) in a unit dose or multi-dose vial for aerosol administration to the lung. The compositions include powder that can be mixed with a diluent to produce a liquid. Surfactants can be used as dispersing agents, solubilizing agents, and spreading agents. Some examples of surfactants are: PEG (polyethylene glycol) 400; Sodium lauryl sulfate sorbitan laurate, sorbitan palmitate, sorbitan stearate available under the tradename Span, (20-40-60 etc.); polyoxyethylene (20) sorbitan monolaurate, polyoxyethylene (20) sorbitan monopalmitate, polyoxyethylene (20) sorbitan monostearate available under the tradename Tweens (polysorbates, 20-40-60 etc.); tyloxapol; propylene glycol; and Benzalkonium chloride. Contemplated surfactants include any compound or agent that lowers the surface tension of a composition. The HLB (hydrophile-lipophile-balance) is used to describe the characteristics of a surfactant. The system consists of an arbitrary scale to which HLB values are experimentally determined and assigned. If the HLB value is low, the number of hydrophilic groups on the surfactant is small, which means it is more lipophilic (oil soluble). An HLB value of 10 or higher means that the agent is primarily hydrophilic, while an HLB value of less than 10 means it would be lipophilic. For example, Spans have HLB values ranging from 1.8 to 8.6, which is indicative of oil soluble or oil dispersible molecules. Consequently, the oil phase will predominate and a water/oil emulsion will be formed. Tween polysurfactants have HLB values that range from 9.6 to 16.7, which are characteristic of water-soluble or water dispersible molecules. Therefore, the water phase will predominate and oil/water emulsions will be formed. Emulsifying agents are surfactants that reduce the interfacial tension between oil and water, thereby minimizing the surface energy through the formation of globules. Wetting agents, on the other hand, aid in attaining intimate contact between solid particles and liquids.

The pH of the formulation is an important feature for aerosolized cardioprotectant delivery. When the aerosol is either too acidic or too basic, it can cause bronchospasm and cough. Although the safe range of pH is relative and some patients may tolerate a mildly acidic aerosol, others will experience bronchospasm. Any aerosol with a pH of less than 4.5 typically induces bronchospasm. Aerosols with a pH between 4.5 and 5.5 will cause bronchospasm occasionally. Any aerosol having pH greater than 7.5 is generally to be avoided as the body tissues are unable to buffer alkaline aerosols. Aerosol with controlled pH below 4.5 and over 7.5 result in lung irritation accompanied by severe bronchospasm cough and inflammatory reactions. Consequently the cardioprotectant aerosol formulation is adjusted to pH between 4.5 and 7.5 with preferred pH range from about 5.5 to 7.0. Most preferred pH range is from 5.5 to 6.5.

Preferred solutions for nebulization of cardioprotectants which are safe and have airway tolerance typically have a total osmolality between 50 and 550 mOsm/kg with a range of chloride concentration of between 31 mM and 300 mM. The given osmolality controls bronchospasm, the chloride concentration, as a permeant anion, controls cough. In this regard the chloride anion can be substituted with bromine or iodine anions, since both are permeant anions. In addition, bicarbonate may be wholly or partially substituted for chloride ion. Normal saline (NS) contains 154 mM of chloride whereas 31 mM of chloride corresponds to about 0.2 normal saline.

### EXAMPLES

The present invention, thus generally described, will be understood more readily by reference to the following examples, which are provided by way of illustration and are not intended to be limiting of the present invention.

### Isolation of Single Guinea-Pig Ventricular Myocytes

Myocytes were isolated enzymatically from guinea-pig ventricle as previously described (Powell et al, 1980; Mitchell et al, 1984). Briefly, male guinea-pigs were killed by cervical dislocation following stunning. Myocytes were isolated after perfusion of the heart with a physiological salt solution containing reduced calcium and 0.8 mg/mL of collagenase Type 1 (Worthington Biochemicals). Cells were stored at room temperature in Dulbecco's MEM (Life Technologies, Scotland) and used for electrophysiological investigation on the day of preparation.

Recording of Action Potentials from Single Ventricular Myocytes.

Action potentials were recorded from single guinea-pig ventricular myocytes using the standard control conditions and criteria as adopted by CCTT Ltd. (see http://www.cctt.ltd.uk/ap1.htm). Briefly, action potentials were recorded using conventional glass microelectrodes filled with 1 M KMeSO₄ and 10 mM KCl. Electrode resistances were in the range of 40 to 60 MΩ. An Axoclamp 2B (Axon Instruments Inc) amplifier was used for electrical recording. Action potentials were continuously stimulated by application of a brief (1-2 ms) current stimuli applied via the intracellular microelectrode in ‘bridge’ mode. Following penetration of the cell with the microelectrode, a stabilisation period of at least 5 min was allowed before recordings were started. The frequency of stimulation was 1 Hz. Recorded signals were digitised at a sampling frequency of 5 kHz and stored for subsequent off line analysis. Action potential signals were continuously monitored online using an oscilloscope (Tektronix digital storage oscilloscope) to check for development of effects of dexrazoxane. All recordings are made at 36±1° C.

### Action Potential Recording Criteria Amplitude and Resting Potential

The stability of several parameters of the action potential profile (resting potential, action potential amplitude and action potential duration) was ensured before recordings were commenced. Resting potential was measured over a 5-10 ms period before the stimulus artefact and values for the resting potential falling between −75 to −85 mV were taken as normal. Action potential amplitude was measured following the stimulus artefact and values falling between 110 to 130 mV were taken as normal.

### Statistics

All data are presented as mean ±SEM. The effect of dexrazoxane on action potential characteristics was assessed using Student's t-test (MicroSoft Excel) and P<0.05 was taken to indicate statistical significance.

### Action Potential Duration

Action potential duration was measured from the peak amplitude of the action potential to 90% repolarisation level (APD₉₀), and values for APD₉₀ falling between 150 to 300 ms were taken as normal. Parameters measured in this study were action potential duration at 40, and 90% repolarisation levels (APD₄₀ and APD₉₀) using Clampfit 9.0.

For each cell, APD₄₀ and APD₉₀ values were calculated in the absence (control) and presence of a drug. As an index of ‘Triangulation’ APD₉₀-APD₄₀, (APD₉₀₋₄₀ ms) in the absence (APD₉₀₋₄₀ control) and presence of dexrazoxane (APD₉₀₋₄₀ drug) were calculated. This was normalised to the starting (control) APD₉₀ to express APD₉₀₋₄₀ duration as a % of control APD₉₀. Calculations were made for each cell individually; the values for each drug concentration were then averaged.

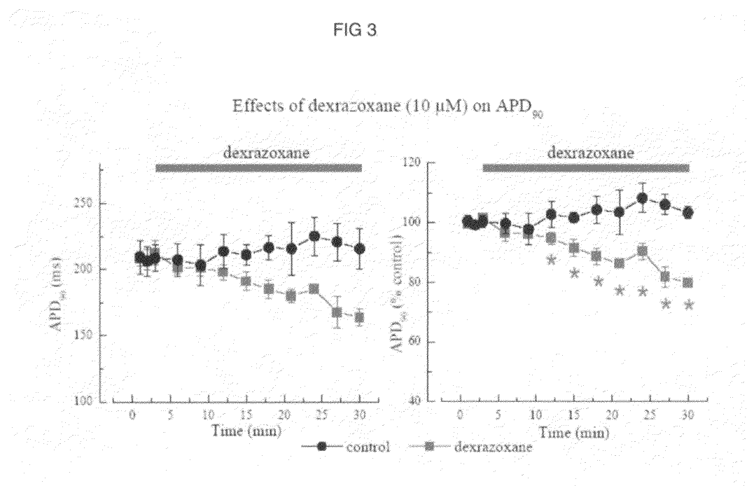

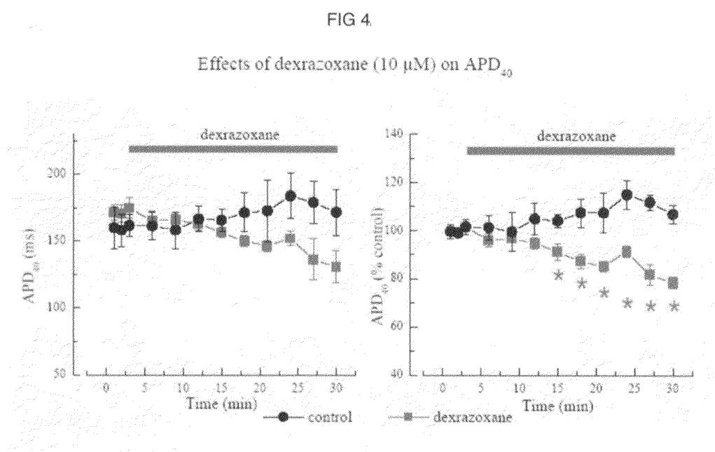

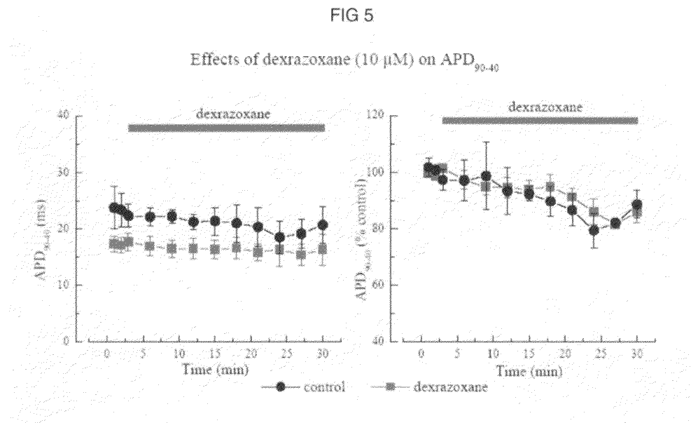

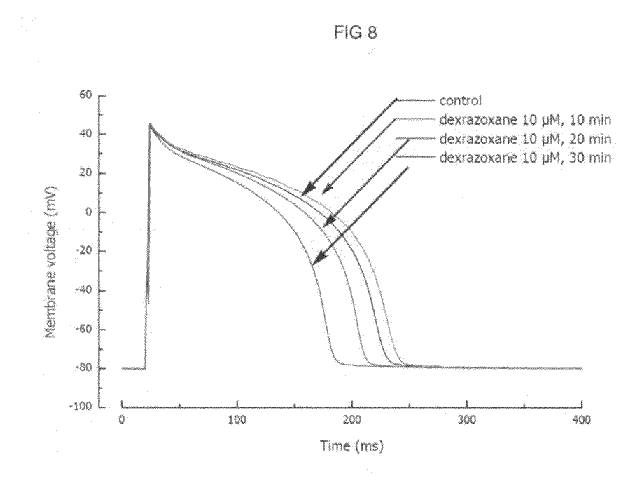

Mean data showing the effect of dexrazoxane (10 μM) on action potential duration (APD₉₀) is shown in FIG. 3. The data shows that perfusion with dexrazoxane (27 minutes) caused an 18±3% shortening of APD₉₀ from 212±9 ms to 168±12 ms (n=4, P<0.005). The addition of dexrazoxane to the superfusion solution caused an 18±4% a decrease in APD₄₀ from 175±8 ms to 136±15 ms (n=3, P<0.005, see FIG. 4). Superfusion of dexrazoxane did not significantly alter the APD₉₀₋₄₀ duration which is taken as an index of triangulation (see FIG. 5). Accordingly, time control data show that under control condition there was no change in either APD₉₀ (FIG. 3), APD₄₀ (FIG. 4), or in APD₉₀₋₄₀ (FIG. 5). Furthermore, FIG. 8 shows typical action potential records taken from a single ventricular myocytes under control conditions and in the presence of dexrazoxane (10 μM) conditions) shortened action potential duration.

### Measurement of Unloaded Cell Shortening

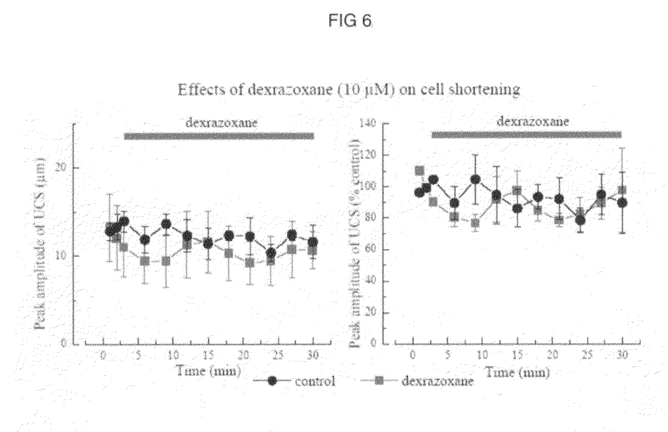

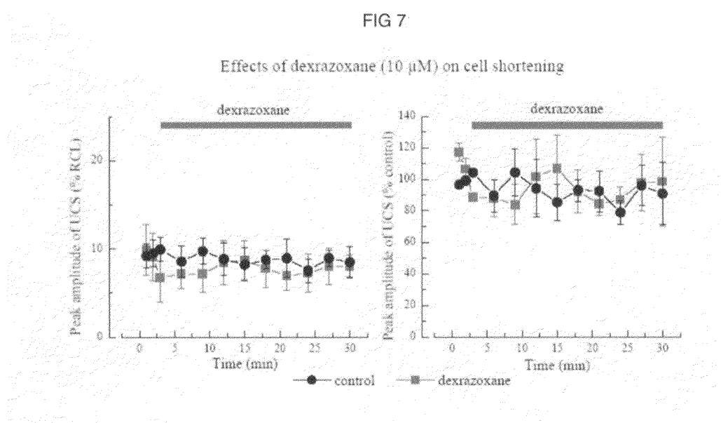

Measurement of unloaded cell shortening (UCS) of single ventricular myocytes was achieved using a video-edge detection system with a temporal resolution of 4.2 ms (SoftEdge™, IonWizard Analysis Software, IonOptix Corp). Peak amplitude UCS is expressed as either absolute contraction (μm) or normalised and therefore expressed as a percentage of resting cell length (% RCL). Mean data for the peak amplitude of action potential driven unloaded cell shortening (UCS) measured in single ventricular myocytes is shown in FIG. 6. Application of dexrazoxane (10 μM, 27 minutes) did not significantly alter the amplitude of UCS expressed as absolute cell shortening (control, 9.4±3.4 μm; dexrazoxane 10.7±3.2 μm, n=3, P=ns; the left panel of FIG. 6 shows contraction in μm while the right panel shows percentage change from control). Similar findings as depicted in FIG. 6 were made when shortening was expressed as percentage of resting cell length as in FIG. 7 (control, 8.5±2.7% RCL; dexrazoxane 8.0±2.0% RCL, n=3, P=ns; the left panel of FIG. 7 shows shortening as percentage of cell length while the right panel shows percentage change from control).

### Test Substance and Solution Formulation

Composition of bath solution: Bath solution was prepared daily. The composition of the bath solution was (mM): NaCl 125; NaHCO₃ 25; KCl 5.4; CaCl₂ 1.8; MgCl₂ 1.0; NaH₂PO₄ 1.2; D-glucose 5.5; pH 7.4 when bubbled with 95% CO₂ and 5% O₂ mixture.

Test substance and formulation: The test substance (dexrazoxane) was made up as a stock solution in H₂O (101M) and then diluted into the bath solution to prepare the test substance perfusion solution. This was done daily. The initial test concentration for dexrazoxane was 10 μM.

In conclusion, although the number of cells tested was small these preliminary data show that under these conditions application of dexrazoxane (10 μM) to single ventricular myocytes caused shortening of action potential duration and did not significantly alter the amplitude of contraction.

### In Vivo Cardiac Electrophysiologic and Antiarrhythmic Effects of Dexrazoxane

The ability of dexrazoxane to alter cardiac electrophysiologic parameters and to terminate atrial arrhythmia is assessed in a pentobarbital-anesthetized canine model of atrial flutter. In this model, atrial flutter is induced following a Y-shaped surgical lesion comprised of an intercaval incision and a connecting incision across the right atrium. Bipolar epicardial electrodes are placed on the inferior vena cava and right atrium for atrial pacing, recording local atrial activation, and for measurement of atrial excitation threshold (AET) and atrial relative (ARRP at 2×AET) and effective refractory periods (AERP at 10×AET). Bipolar electrodes also are sutured to the left ventricle for measurement of ventricular excitation threshold (VET) and ventricular relative (VRRP at 2×VET) and effective refractory periods (VERP at 10×VET). The following cardiac electrophysiologic parameters also are measured before dexrazoxane administration and at the termination of the study: AH interval, an index of AV nodal conduction; HV interval, an index of His-ventricular conduction time; PA interval, an index of intra-atrial conduction; and H-EG interval, an index of ventricular conduction; and paced ECG QT interval. AV nodal functional refractory period (AVNFRP) and SA conduction time (SACT) are determined by the introduction of atrial extrastimuli during sinus rhythm, and the monitoring of ventricular response. Electrocardiographic intervals are determined during sinus rhythm; the rate-corrected ECG QTc interval is calculated as: QTc=QT (msec)/R−R (sec). Dexrazoxane is administered as cumulative i.v. doses of 1, 3 and 10 mg/kg, with each dose administered as an intravenous bolus in a vehicle of PEG-200. Additionally, ADR-925 is administered in equal amounts, in order to determine the active moiety of dexrazoxane.

Sustained atrial flutter is initiated in three dogs by electrical burst pacing (6-20 Hz) of the atria; atrial rates range from 440-530 cycles/min. Intravenous bolus administration of PEG-200 vehicle alone has no effect on the atrial arrhythmia in all three animals. Intravenous bolus administration of 3 mg/kg of dexrazoxane (and ADR-925=>side-by-side) in two animals, and 10 mg/kg of dexrazoxane (and ADR925) in the third animal is preformed.

### Dexrazoxane Protective Effects

In this procedure [Lawson, J. Pharmacol. Exper. Therap., Vol. 160, pages 22-31 (1968)], the test substance dexrazoxane is administered i.p. (100 mg/kg) to a group of three mice thirty minutes before exposure to deep chloroform anesthesia and observed during the ensuing 15-minute period. Absence of EKG recorded cardiac arrhythmias and heart rates above 200 beats per minute present (usual=400-480 beats per minute) in none or only one (<2) of three animals indicates significant protection.

### Further Anti-Arrhythmic Dexrazoxane Studies

Animals are implanted with a 25 mg pellet of DOCA and maintained on the 1% saline as their drinking fluid. The animals are anesthetized with Avertin (tribromoethanol, 2.5%), and prepared for the recording of blood pressure and the EKG. In addition, the animal's left jugular vein is also cannulated with PE-50 tubing. The tubings are filled with saline and exteriorized dorsally between the animal's shoulders. Isoproterenol (a proarrhythmic agent) is administered s.c. at a dose of 150 μg/kg and an arrhythmia was allowed to develop for five minutes. After this time, either propranolol (control) or dexrazoxane is given as an intravenous bolus at one-half of the effective s.c. dose. Continuous blood pressure, heart rate and EKG readings are obtained. Additional Avertin is given as needed to maintain anesthesia. The hearts of the animals are perfused with buffered formalin immediately after the onset of ventricular fibrillation or one hour after the administration of isoproterenol.

### Effects of Doxorubicin and Dexrazoxane on the Isolated Perfused Heart.

Perfusion studies of doxorubicin and dexrazoxane in the isolated, perfused guinea pig heart were performed similarly as above. The following parameters were recorded continuously: electrocardiogram (ECG), monophasic action potentials (MAP), perfusion pressure, left ventricular pressure and coronary flow. These effects could be related to some modifications of cardiac electrophysiology (changes in PR, QRS, QT and RR intervals, proarrhythmic effects, Torsade de pointes . . . ), of coronary resistance and/or of cardiac contraction or inotropism (contractility or relaxation troubles).

Stock solutions (10⁻²M, 3×10⁻²M) in H₂0 for each drug were prepared each day of the experiment and a dilution (1/1000) done in the perfusion buffer in order to get the appropriate concentrations:

### D1: 10⁻⁵M

### D2: 3×10⁻⁵M

The buffer used in the experiment was KREBS-HENSELEIT buffer (Sigma K-3753) to which was added sodium bicarbonate (Sigma S5761) 25 mM and calcium chloride dihydrate (Sigma C8106) 1.8 mM.

Measured Parameters

### —Electrophysiology

The ECG (2 electrodes: on the apex and closed to the right auricular) and the MAP (electrode on the epicardium) were recorded. PR, QRS, QT and RR were measured and QTc (Fridericia; QTc=QT/(RR) ⅓ in ms) was calculated.

### —Coronary Perfusion

The coronary flow (mL/min) is indicated by the perfusion pump. In this model, the perfusion pressure was constant.

### —Cardiac Contractility (Inotropism)

Diastolic pressure (mmHg), systolic pressure (mmHg), differential pressure (syst-diast) (mmHg), mean pressure (mmHg), dP/dt min (mmHg/sec) dP/dt max (mmHg/sec) and cardiac frequency (beat per minute: BPM) were measured. A modification of the inotropism can be a problem of cardiac contractility (if modified systolic pressure and dP/dt max) or relaxation (if modified diastolic pressure and dP/dt min). From the left ventricular pressure, the following were calculated: heart rate (frequency of cardiac contractions), dP/dt min and dP/dt max. An acquisition unit MP150WSW with Acknowledge software (Biopac, USA) was used to continuously record all the signals (perfusion pressure, coronary flow, left ventricular pressure, ECG and MAP) through a PC computer.
In each group (n=4 hearts), increasing concentrations of compound were infused according to the following design:

Stabilisation (Krebs-Henseleit Buffer, during 45 minutes)

| | | |
|---|---|---|
| D0: Baseline: Vehicle (Krebs) | T0 to T5 | min |
| D1: compound at 10⁻⁵ M | T5 to T50 | min |
| D2: compound at 3 × 10⁻⁵ M | T50 to T95 | min |
| D3: Krebs-Henseleit Buffer (recovery) | T95 to T105 | min |

For each group, the data are expressed as mean ±S.E.M, % of variation compared to D0 and delta variation from D0.
PR, QRS, QT and RR are measured on the same period but on a signal which results from 5 cycles.

On all the curves, the mean value is measured on 30 seconds in the last minute of perfusion for the following parameters:

perfusion pressure

coronary flow

mean left ventricular pressure

maximum left ventricular pressure

minimum left ventricular pressure

dP/dt min

dP/dt max

heart rate

APD90

The data for doxorubicin, dexrazoxane, and doxorubicin plus dexrazoxane at the 30 minute perfusion time point are given in Table 3. Observed was a marked increase in LVEDP from doxorubicin perfusion. Addition of dexrazoxane to the perfusion did not contribute to the doxorubicin induced increase in LVEDP. QTcF was increased following doxorubicin perfusion however, the increase observed at both doxorubicin doses was reversed by the addition of dexrazoxane.

### Calcium Transients and Action Potential Duration in Isolated Guinea Pig Myocytes

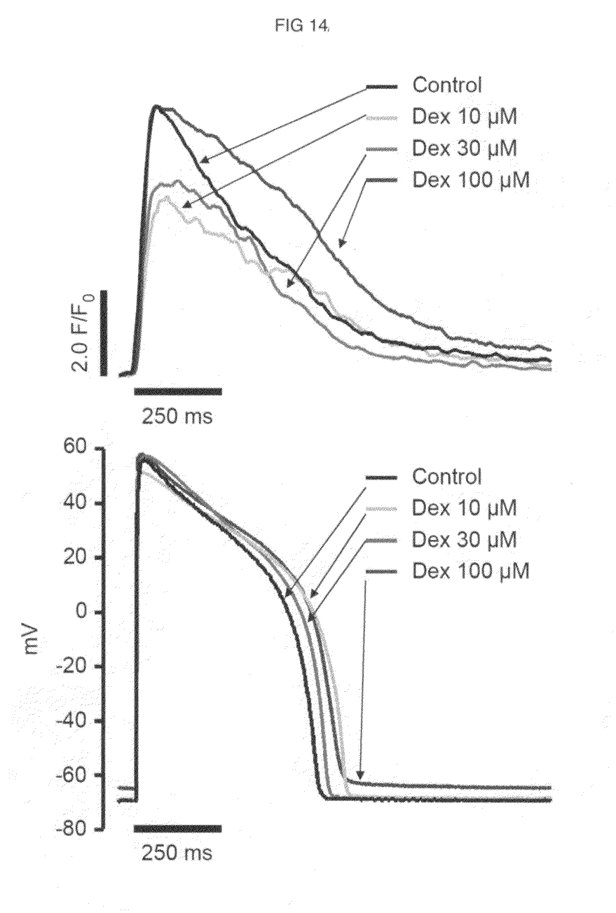

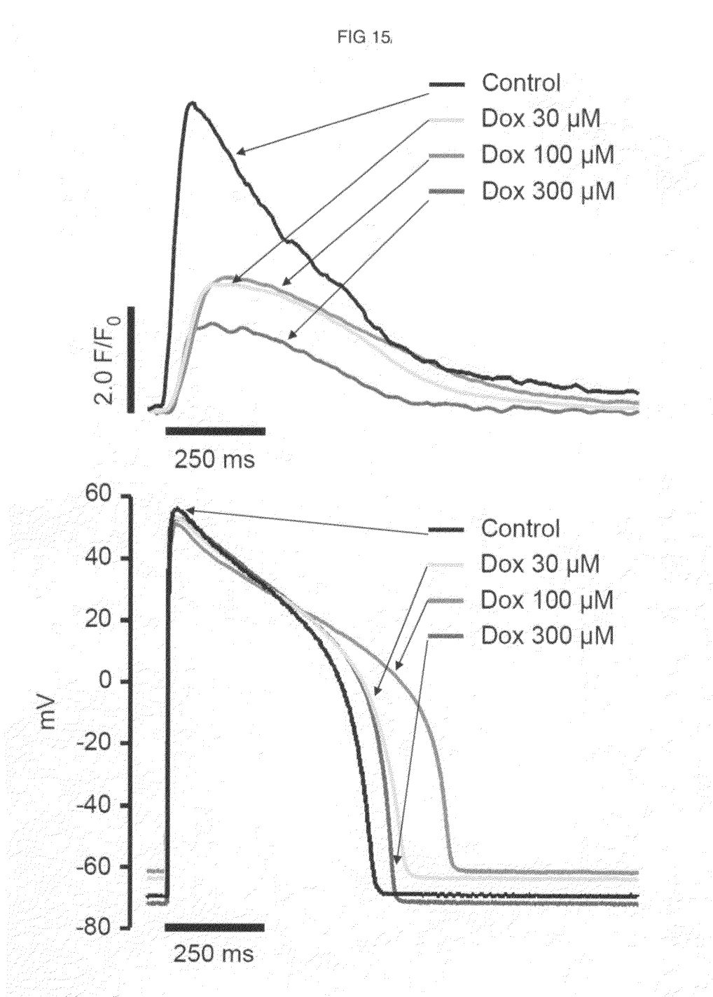

Calcium transients were measured during activation in isolated guinea pig myocytes. Expressed as the fluorescence ratio vs. time (ms), representative traces were recorded in myocytes untreated and treated with either 10 μM dexrazoxane, 30 μM dexrazoxane, 100 μM dexrazoxane, or untreated (FIG. 14 top) or with either 30 μM doxorubicin, 100 μM doxorubicin, 300 μM doxorubicin, or untreated control (FIG. 15 top). Action potential amplitude vs. time (ms) was measured in the myocytes under similar conditions and drug concentrations for dexrazoxane (FIG. 14 bottom) and for doxorubicin (FIG. 15 bottom). Table 4 shows the tabulated effects of doxorubicin and dexrazoxane on calcium transients and action potential properties as well as the effects of perfusion with 100 μM doxorubicin in combination with 30 μM dexrazoxane. The data indicate that dexrazoxane causes a dose proportional increase in calcium transients without inducing a change in action potential duration. This increase in calcium transient without an action potential increase indicates an effect on calcium cycling proteins. Doxorubicin shows a dose related decrease in calcium transients and a profound prolongation of action potential duration. The combination of dexrazoxane and doxorubicin corrects for the increase in action potential duration.

### Synthesis of Dexrazoxane

Propane-1,2-diamine tetra-acetic acid (9 g), and formamide (250 mL) are heated at 110° C. together under reduced pressure (ca 100 mm.) for 2 hours. The heat is then raised to 160° C. and stirred for 3 additional hours. The reaction is cooled until precipitate forms and product is filtered and then washed with formamide and ethanol. Further purification is performed by column chromatography (C₁₁H₁₆N₄O₄, MH+ 269.3, m.p. 191-197° C.).

### Synthesis of Dexrazoxane Derivatives

Useful synthetic routes are described in U.S. Pat. No. 3,941,790.

A diamino alkane (such as diaminoethane, diaminocyclobutane, or propane-1,2diamine) tetra-acetic acid, and formamide are heated at 110° C. together under reduced pressure for 1-2 hours. The heat is then raised to 150° C. and stirred for 2-3 additional hours. The reaction is cooled, filtered and then washed with ethanol.

Compounds with variable terminal rings are synthesized as follows: the nitrogen-containing heterocyclyl with the desired terminal ring structure (such as 1,3dimethylpiperazine-2,6-dione or morpholin-2-one) are added to a solution containing an alkyldihalide (such as 1,2-dichloropropane) in THF and the solution is heated until complete. Final compounds are identified by GC or LCMS and purified by column chromatography.

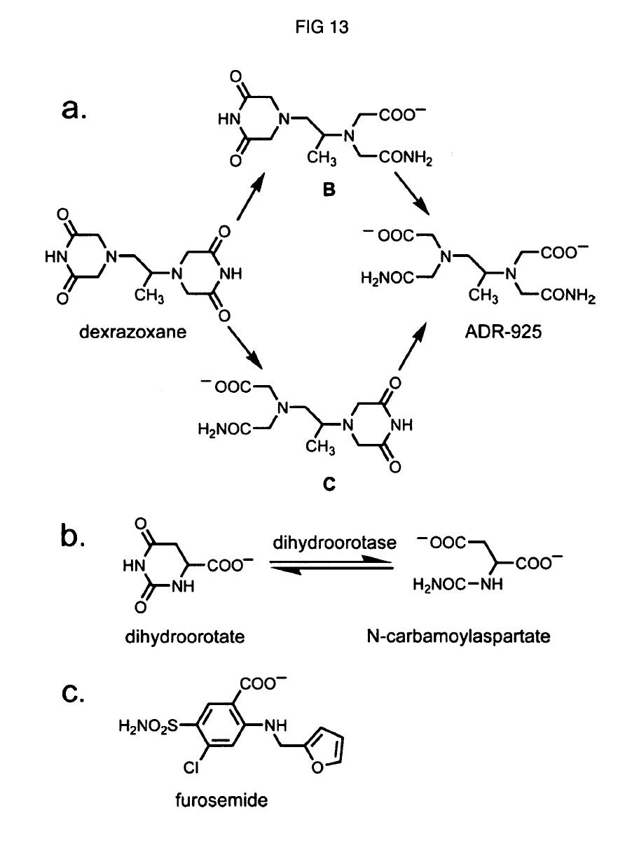

Synthesis of N,N′-[(1S)-1-methyl-1,2-ethanediyl]bis[N-(2-amino-2-oxoethyl)glycine] (ADR-925) and Monocyclic Analogs (B and C, see FIG. 13):

The above are prepared by hydrolyzing 5 mg/ml dexrazoxane with NaOH (40 μl/ml of 1 M NaOH) at 25° C. for 40 min and quenching the reaction with HCl (45 μl/ml of 1 M HCl) to pH 3 as described previously. Under these conditions a mixture of dexrazoxane, B, C, and ADR-925 is produced. Dexrazoxane is efficiently removed from the reaction mixture by loading 500 μl of the mixture on a Sep-Pak Plus C₁₈ cartridge (Waters, Mississauga, ON, Canada) and eluting with 2% (v/v) methanol at a flow rate of 1 ml/min. Although dexrazoxane is highly retained on the cartridge, B, C, and ADR-925 eluted together and are collected at elution volumes between 1.5 and 2.5 ml. HPLC analysis confirmed that dexrazoxane is not detectable in this fraction. This 1-ml fraction, pH 6, is loaded on three Sep-Pak Accell Plus QMA (Waters) ion exchange cartridges connected in series and eluted with 2% (v/v) methanol at a flow rate of 5 ml/min. Fractions containing B are collected at elution volumes between 3 and 4.5 ml, and those containing C between 5 and 9 ml. The B fraction contains less than 0.1 mol % and 0.01 mol % of C and ADR-925, respectively. The C fraction contains less than 0.1 mol % B and 0.05 mol % of ADR-925, respectively. These fractions are brought to pH 2 with 5 M HCl and evaporated to dryness under a stream of nitrogen, stored at 80° C., and reconstituted in water just before use. Neither of these fractions contain detectable amounts of dexrazoxane (<0.001 mol %). Typical yields of B and C are 10 and 6 μg, respectively. Synthesis of cyclic dexrazoxane derivatives:

Preparation of (±) trans-1,2-bis(3,5-dioxopiperazin-1-yl)cyclobutane (U.S. Pat. No. 5,438,057):

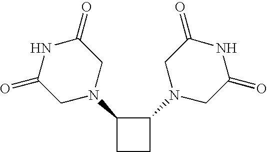

Trans-1,2-Diaminocyclobutane tetra-acetic acid monohydrate (100 g) (melting point 234°-235° C.; prepared in 56% yield by the method of Dwyer and Garvan, J. Amer. Chem. Soc., 1959, 81, 2956), is heated with 400 ml of formamide under nitrogen at reduced pressure at 100°-110° C. for 1 hour and then at 150°-155° C. for 4 hours. The brown solution is evaporated under reduced pressure at 80°-90° C. and the residue taken up in 120 ml of methanol and cooled in a refrigerator overnight. Filtration, follows by washing with cold methanol and vacuum drying at 65° C. Gives a 69% yield of (±) trans1,2-bis(3,5-dioxopiperazin-1-yl)cyclobutane, melting point is reported at 257°-259° C.

Prodrugs of the compositions of the present invention, particularly of (IB) (ADR925), are provided in Table 1.

The following analogs identified under formula (IB) are commercially available from Acros Organics:

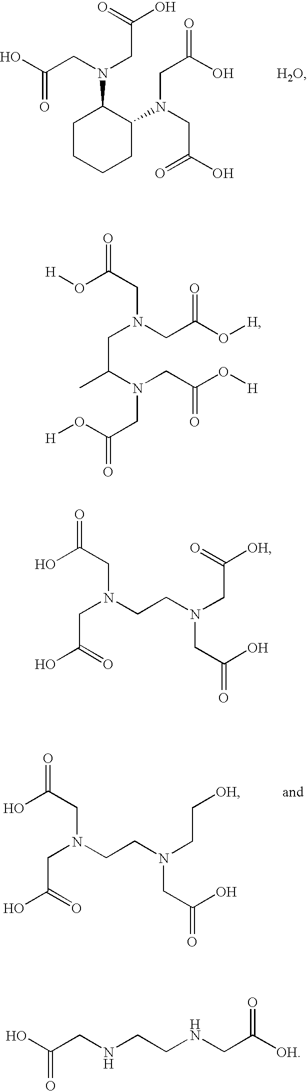

Dexrazoxane, ADR-925, and mono-cyclic metabolites B and C shown in FIG. 13 are synthesized as described above and according to Scheme 1.

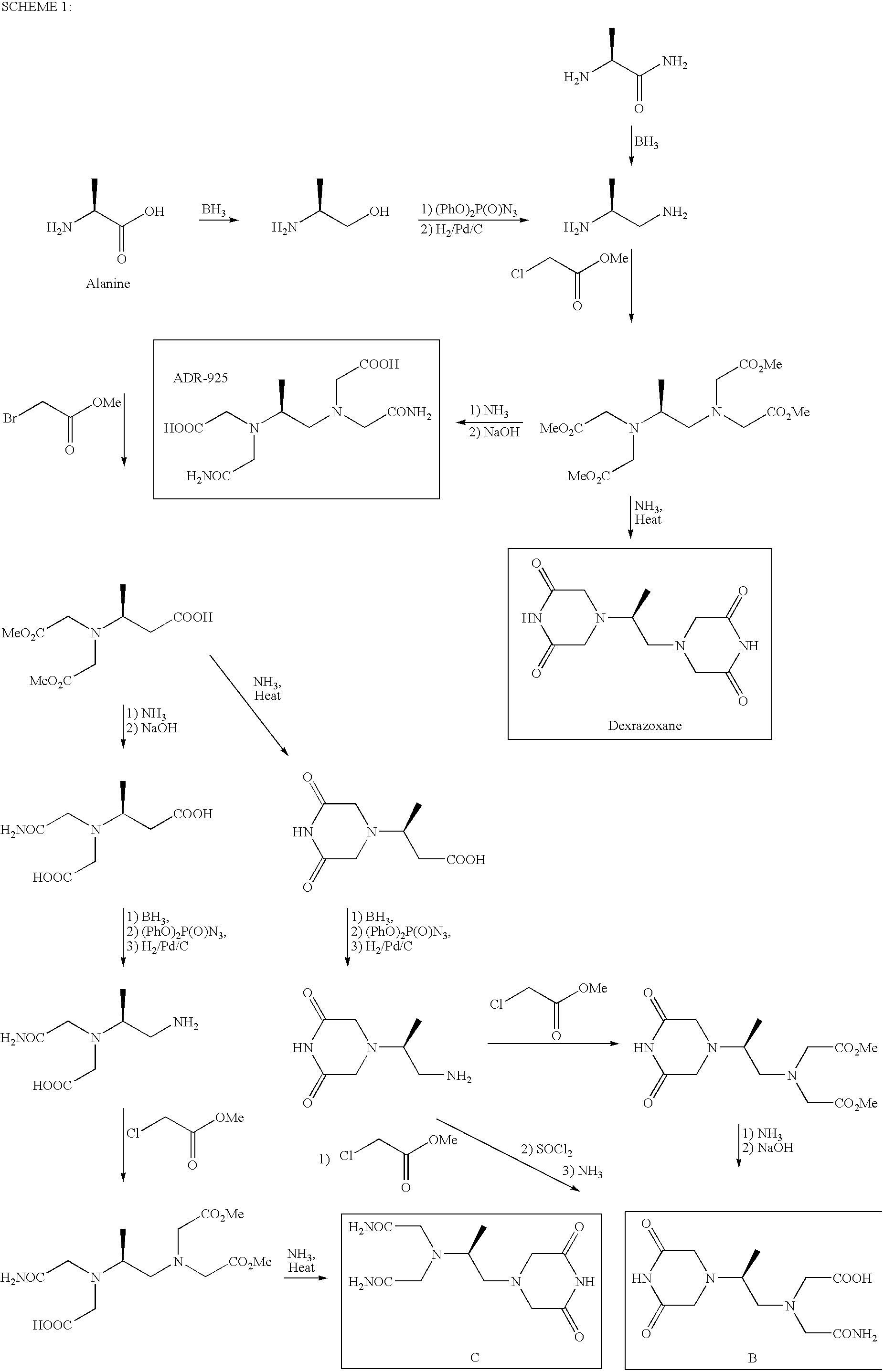

Additionally, other analogs and prodrugs of the compounds, particularly those falling under Formula (IA), (IB), (IC), and prodrugs of (IB), particularly prodrugs of ADR925 are synthesized by modifying useful intermediates (*) depicted in Scheme 1, which will be readily apparent to those skilled in the art. For instance, Examples 1315 in Table 1 are prepared from (S)-propane-1,2-diamine (depicted in top right of Scheme 1*) by substituting with ethyl 2-chloroacetate in place of methyl 2chloroacetate and subsequently mild treatment with ammonia (no NaOH) in the following step yields the desired/separable products.

Additionally, compounds of formula (IA), (IB) and (IC) are synthesized according to the following schemes:

Compounds and prodrugs of formula (IB) are synthesized according to Scheme 2.

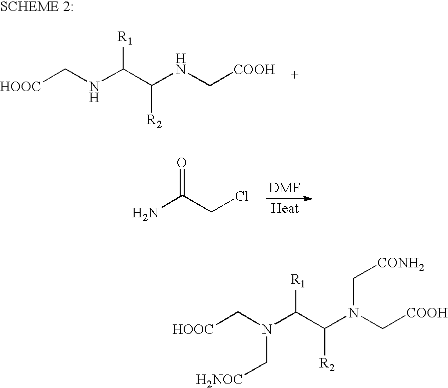

Additional compounds and prodrugs of formula (IB) are synthesized according to Scheme 3.

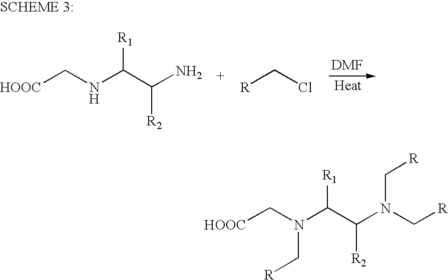

Compounds of formula (IC) are synthesized according to Scheme 4.

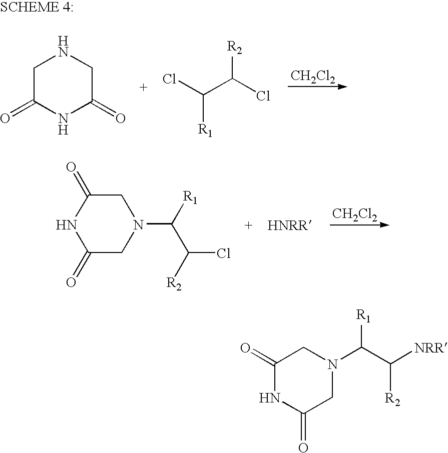

Compounds of formula (IA) are synthesized according to Scheme 5.

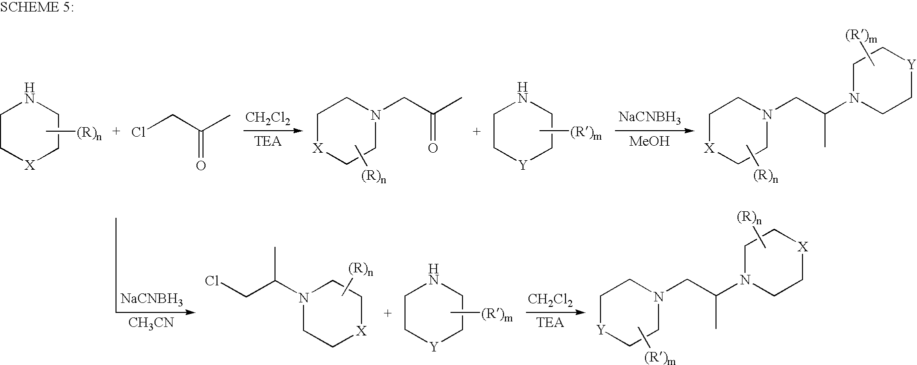

Further derivatives of dexrazoxane are provided in Table 2:

The mechanistic profile for Example 25 is provided in Scheme 6:

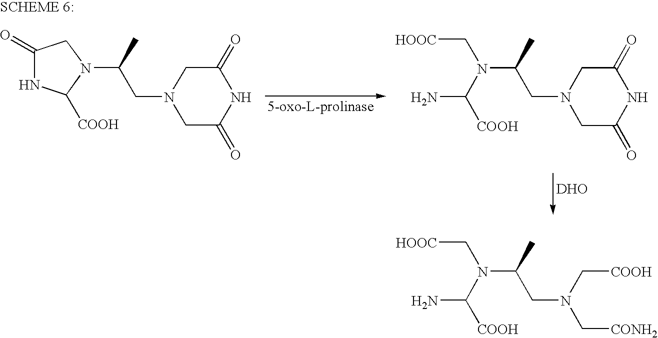

While the invention has been described with respect to specific examples including presently preferred modes of carrying out the invention, those skilled in the art will appreciate that there are numerous variations and permutations of the above described systems and techniques that fall within the spirit and scope of the invention.

The contents of all of the above cited patents, patent applications and journal articles are incorporated by reference as if set forth fully herein.

TABLE 1
ADR-925 Prodrug Examples

| | | | | Metabolic |
|---|---|---|---|---|
| Example | Structure | MW | Formula | Pathway |
| 1 | 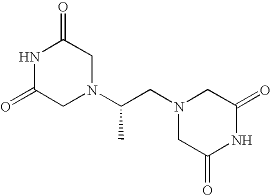 | 268.27 | C11H16N4O4 | DHO |
| 2 | 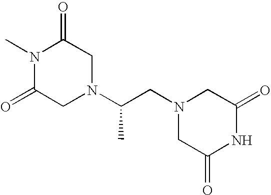 | 282.13 | C12H18N4O4 | CytochromeP450; DHO |
| 3 | 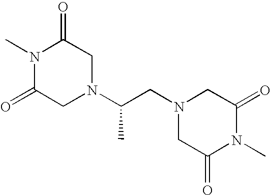 | 296.32 | C13H20N4O4 | CytochromeP450; DHO |
| 4 | 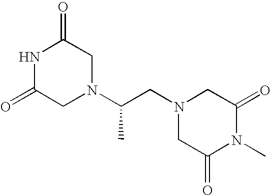 | 282.13 | C12H18N4O4 | CytochromeP450; DHO |
| 5 | 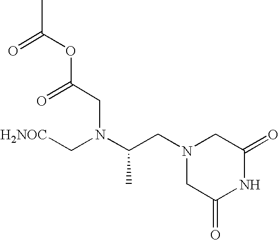 | 328.32 | C13H20N4O6 | Acid-catalyzedhydrolysis;DHO |
| 6 | 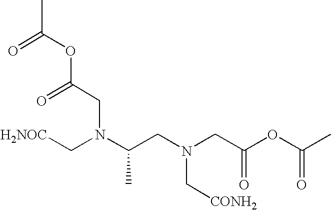 | 388.37 | C15H24N4O8 | Acid-catalyzedhydrolysis;DHO |
| 7 | 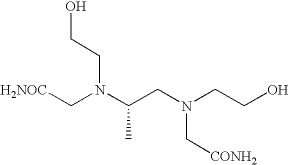 | 276.33 | C11H24N4O4 | CytochromeP450; |
| 8 | 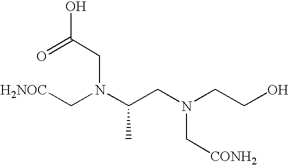 | 290.32 | C11H22N4O5 | CytochromeP450; |
| 9 | 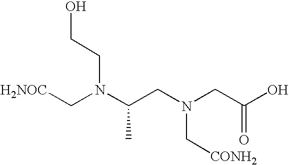 | 290.32 | C11H22N4O5 | CytochromeP450; |
| 10 | 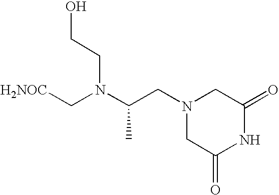 | 272.3 | C11H20N4O4 | CytochromeP450; DHO |
| 11 | 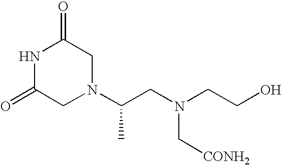 | 272.3 | C11H20N4O4 | CytochromeP450; DHO |
| 12 | 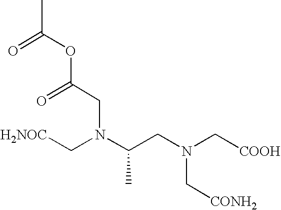 | 346.34 | C13H22N4O7 | Acid-catalyzedhydrolysis |
| 13 | 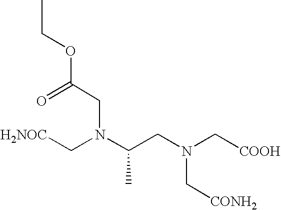 | 332.35 | C13H24N4O6 | Esterase |
| 14 | 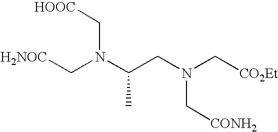 | 332.35 | C13H24N4O6 | Esterase |
| 15 | 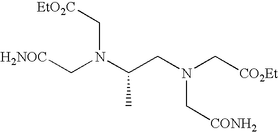 | 360.41 | C15H28N4O6 | Esterase |
| 16 | 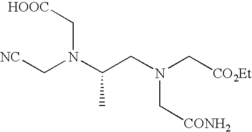 | 314.34 | C13H22N4O5 | Esterase,AcidCatalysis |
| 17 | 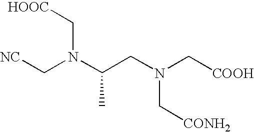 | 286.28 | C11H18N4O5 | AcidCatalysis |
| 18 | 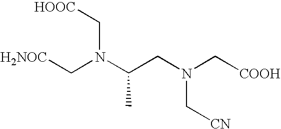 | 286.28 | C11H18N4O5 | AcidCatalysis |
| 19 | 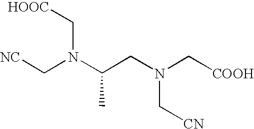 | 268.27 | C11H16N4O4 | AcidCatalysis |
| 20 | 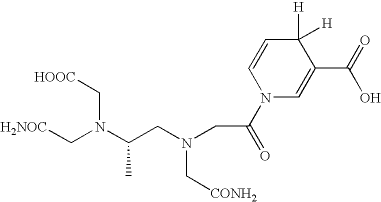 | 411.41 | C17H25N5O7 | Esterase |
| 21 | 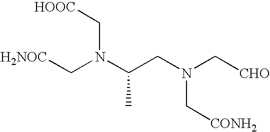 | 288.3 | C11H20N4O5 | CytochromeP450 |
| 22 | 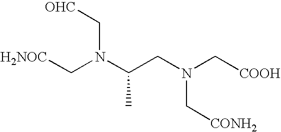 | 288.3 | C11H20N4O5 | CytochromeP450 |
| 23 | 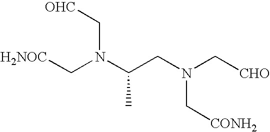 | 272.3 | C11H20N4O4 | CytochromeP450 |
| 24 | 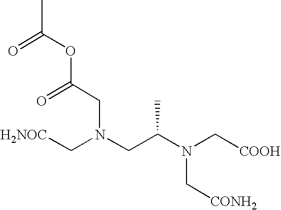 | 346.34 | C13H22N4O7 | Acid-catalyzedhydrolysis |

TABLE 2
Dexrazoxane Derivatives

| Example | Structure | MW | Formula |
|---|---|---|---|
| 25 | 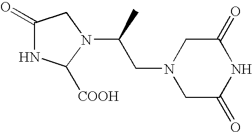 | 284.27 | C11H16N4O5 |
| 26 | 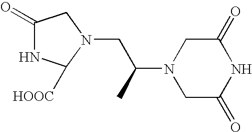 | 284.27 | C11H16N4O5 |
| 27 | 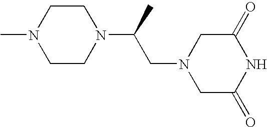 | 254.33 | C12H22N4O2 |
| 28 | 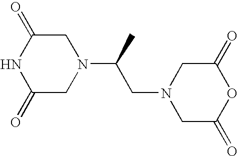 | 269.25 | C11H15N3O5 |
| 29 | 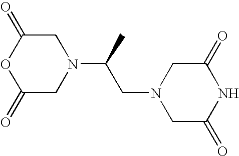 | 269.25 | C11H15N3O5 |
| 30 | 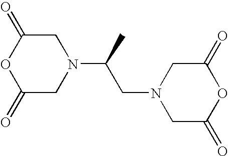 | 270.24 | C11H14N2O6 |
| 31 | 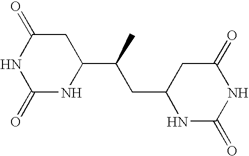 | 268.27 | C11H16N4O4 |
| 32 | 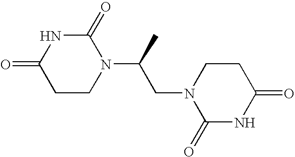 | 268.27 | C11H16N4O4 |
| 33 | 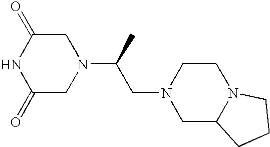 | 280.37 | C14H24N4O2 |
| 34 | 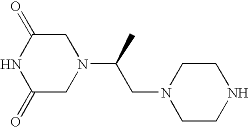 | 240.3 | C11H20N4O2 |
| 35 | 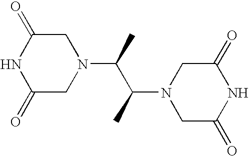 | 282.3 | C12H18N4O4 |
| 36 | 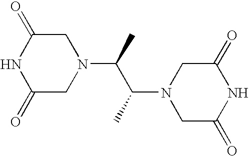 | 282.3 | C12H18N4O4 |
| 37 | 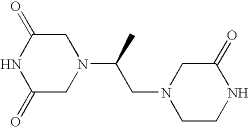 | 254.29 | C11H18N4O3 |

TABLE 3
Doxorubicin and Dexrazoxane Effects on Cardiac Parameters Measured in
Langendorff Guinea Pig Heart Preparations

| | | DOX 10⁻⁵ mM | | DOX 3 * 10⁻⁵ mM | |
|---|---|---|---|---|---|
| | | 30 | | 30 | |
| | Min of perfusion | mean | SEM | Mean | SEM |
| Cor flow | | | | | |
| DEX Alone | ml/min | 14 | 1 | 14.6 | 1.1 |
| DOX Alone | | 13.4 | 1.5 | 14.4 | 1.5 |
| DEX plus DOX | | 11.7 | 1.1 | 15 | 0.6 |
| LVEDP | | | | | |
| DEX Alone | mmHg | 5 | 1 | 5 | 1 |
| DOX Alone | | 7 | 1 | 14 | 5 |
| DEX plus DOX | | 7 | 2 | 16 | 4 |
| LV peak systolic | | | | | |
| pressure | | | | | |
| DEX Alone | mmHg | 94 | 8 | 82 | 7 |
| DOX Alone | | 97 | 9 | 93 | 7 |
| DEX plus DOX | | 108 | 3 | 109 | 3 |
| LV dp/dt max | | | | | |
| DEX Alone | mmHg sec⁻¹ | 1238 | 110 | 1041 | 97 |
| DOX Alone | | 1277 | 175 | 887 | 66 |
| DEX plus DOX | | 1317 | 41 | 1022 | 37 |
| LV dpdt (relax) | | | | | |
| DEX Alone | mmHg sec⁻¹ | −1139 | 93 | −1022 | 90 |
| DOX Alone | | −1173 | 98 | −922 | 44 |
| DEX plus DOX | | −1262 | 51 | −1035 | 31 |
| Cardiac rate | | | | | |
| DEX Alone | Beats/min | 203 | 2 | 200 | 3 |
| DOX Alone | | 217 | 11 | 184 | 10 |
| DEX plus DOX | | 194 | 5 | 174 | 4 |
| QTcF | | | | | |
| DEX Alone | msec | 236 | 3 | 237 | 3 |
| DOX Alone | | 247 | 9 | 272 | 6 |
| DEX plus DOX | | 236 | 3 | 245 | 4 |
| % change in QTc | | | | | |
| DEX Alone | % change | −2.1 | | −1.7 | |
| DOX Alone | | 1.6 | | 11.9 | |
| DEX plus DOX | | −2.1 | | 1.7 | |
| MAP Duration | | | | | |
| DEX Alone | msec | 139 | | 143 | |
| DOX Alone | | 142 | | 162 | |
| DEX plus DOX | | 144 | | 156 | |
| % change in MAP | | | | | |
| duration | | | | | |
| DEX Alone | | 0 | | 0.0 | |
| DOX Alone | | 2.2 | | 13.3 | |
| DEX plus DOX | | 3.6 | | 9.1 | |

TABLE 4
Doxorubicin and Dexrazoxane Effects on Action Potential and Calcium Flux in
Isolated Guinea Pig Myocytes

| | | Doxorubicin | | | Dexrazoxane | | | Dox/Dex |
|---|---|---|---|---|---|---|---|---|
| | Ctrl | 30 μM | 100 μM | 300 μM | 10 μM | 30 μM | 100 μM | 100/30 μM |
| | (n = 13) | (n = 4) | (n = 6) | (n = 7) | (n = 4) | (n = 5) | (n = 4) | (n = 4) |
| Ca²⁺ transients | | | | | | | | |
| Peak amplitude (ΔF/F₀) | 4.9 ± 0.7 | 3.0 ± 0.7 | 3.1 ± 0.5 | 1.6 ± 0.3^{†} | 5.2 ± 0.8 | 5.8 ± 1.0 | 5.1 ± 0.6 | 2.5 ± 0.3** |
| Action potentials | | | | | | | | |
| Resting potential (mV) | −69 ± 2 | −70 ± 3 | −68 ± 3 | −69 ± 2 | −69 ± 0 | −68 ± 2 | −66 ± 1 | −64 ± 1 |
| Peak amplitude (mV) | 116 ± 2 | 120 ± 3 | 119 ± 2 | 122 ± 4 | 119 ± 2 | 121 ± 2 | 124 ± 1^{#} | 111 ± 5 |
| APD₅₀ (ms) | 455 ± 21 | 553 ± 29* | 646 ± 26^{†} | 536 ± 48 | 512 ± 47 | 493 ± 36 | 548 ± 45 | 489 ± 54 |
| APD₉₀ (ms) | 523 ± 19 | 626 ± 28* | 713 ± 32^{†} | 611 ± 48 | 573 ± 43 | 564 ± 33 | 619 ± 39 | 599 ± 45 |

*P < 0.05
**P < 0.01
^{#}P < 0.005
^{†}P < 0.001

1. A method of treating a condition arising from iron or calcium overload comprising administering to a patient in need thereof, an effective amount of a compound of formula (IA), (IB), or (IC): 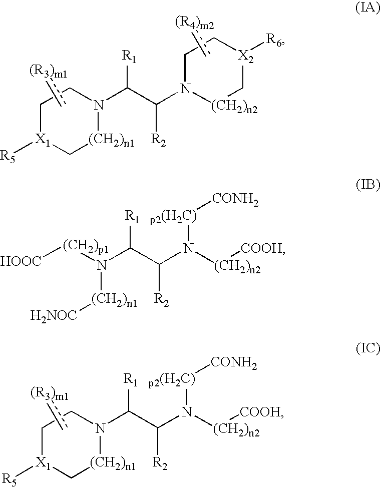
wherein,
X₁ and X₂ are independently selected form CH, N, S, or O;
each R₁ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl;
each R₂ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl; or
R₁ and R₂ are taken together to form a 3-7 membered substituted or unsubstituted carbocyclyl or heterocyclyl group;
each R₃ and R₄ group is independently selected from ═O, ═S, ═NH, H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl, wherein the dotted line represents the optional placement of a double bond; R₅ and R₆ are independently selected from H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl;
m1 and m2 are independently 1, 2, or 3; n1 and n2 are independently 0, 1, or 2; and p1 and p2 are independently 0, 1, or 2;
wherein if X₁ is O or S, then R₅ is absent, and wherein if X₂ is O or S, then R₆ is absent;
or a pharmaceutically acceptable salt, tautomer, stereoisomer, or prodrug thereof;
provided that if R₃ and R₄ are ═O, m1 and m2 are 2, and R₅ and R₆ are H, substituted or unsubstituted carbonyl, substituted or unsubstituted C₁₋₆ alkyl, substituted or unsubstituted C₁₋₆ alkoxy, substituted or unsubstituted C₁₋₆ alkenyl, then R₁ and R₂ are not H, —CH₃, or taken together do not form —CH₂—, —CHCH—, or —CH₂CH₂—.

2. A method according to claim 1 wherein the compound is of formula (IB): 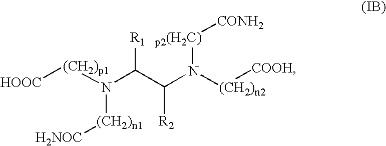
wherein,
R₁ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl;
R₂ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl; or
R₁ and R₂ are taken together to form a 3-7 membered substituted or unsubstituted carbocyclyl or heterocyclyl group;
n1 and n2 are independently 0, 1, or 2; and
p1 and p2 are independently 0, 1, or 2;
or a pharmaceutically acceptable salt, tautomer, stereoisomer, or prodrug thereof.

3. The method of claim 1, wherein the compound has formula (IA).

4. The method of claim 1 wherein the condition is cardiotoxicity.

5. The method of claim 1, wherein the compound has formula (IB) or (IC) and n1, n2, p1, and p2 are 1.

6. A method of treatment comprising administering to a patient in need thereof, an effective amount of a compound of formula (IA), (IB), or (IC): 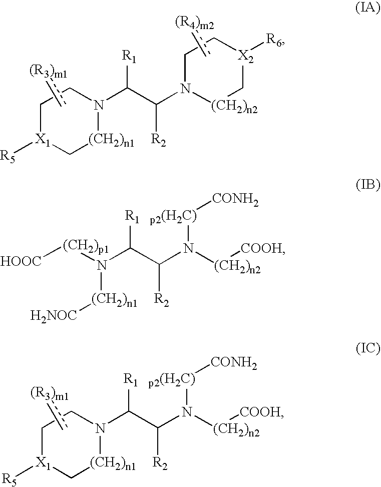
wherein,
X₁ and X₂ are independently selected form CH, N, S, or O;
each R₁ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl;
each R₂ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl; or
R₁ and R₂ are taken together to form a 3-7 membered substituted or unsubstituted carbocyclyl or heterocyclyl group;
each R₃ and R₄ group is independently selected from ═O, ═S, ═NH, H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl, wherein the dotted line represents the optional placement of a double bond; R₅ and R₆ are independently selected from H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl;
m1 and m2 are independently 1, 2, or 3; n1 and n2 are independently 0, 1, or 2; and p1 and p2 are independently 0, 1, or 2;
wherein if X₁ is O or S, then R₁ is absent, and wherein if X₂ is O or S, then R₆ is absent;
or a pharmaceutically acceptable salt, tautomer, stereoisomer, or prodrug thereof; and
an EGFR modulating agent.

7. The method of claim 6 wherein the EGFR modulating agent is selected from the group consisting of trastuzumab, cetuximab, bevacizumab, panitumumab, gefitinib, erlotinib, or combinations thereof.

8. The method of claim 7 wherein the patient has a genetic polymorphism of a calcium cycling protein.

9. The method of claim 6 further comprising administering an anthracycline chemotherapeutic agent.

10. The method of claim 6, wherein the compound has formula (IA).

11. The method of claim 10 wherein the compound is dexrazoxane.

12. The method of claim 1, wherein the compound has formula (IB) or (IC) and n1, n2, p1, and p2 are 1.

13. A method according to claim 6 wherein the compound is of formula (IB): 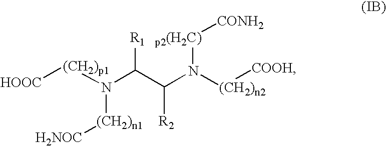
wherein,
R₁ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl;
R₂ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl; or
R₁ and R₂ are taken together to form a 3-7 membered substituted or unsubstituted carbocyclyl or heterocyclyl group;
n1 and n2 are independently 0, 1, or 2; and
p1 and p2 are independently 0, 1, or 2;
or a pharmaceutically acceptable salt, tautomer, stereoisomer, or prodrug thereof.

14. A method of treatment comprising administering to a patient in need thereof via inhalation, an effective amount of a dry powder or liquid aerosol pharmaceutical composition comprising a compound of formula (IA), (IB), or (IC): 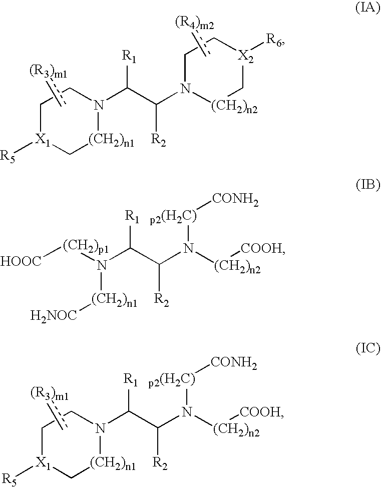
wherein,
X₁ and X₂ are independently selected form CH, N, S, or O;
each R₁ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl;
each R₂ is H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₂₋₆ alkenyl, substituted or unsubstituted C₆₋₁₀ aryl, or substituted or unsubstituted heterocyclyl; or
R₁ and R₂ are taken together to form a 3-7 membered substituted or unsubstituted carbocyclyl or heterocyclyl group;
each R₃ and R₄ group is independently selected from ═O, ═S, ═NH, H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl, wherein the dotted line represents the optional placement of a double bond; R₁ and R₆ are independently selected from H, halogen, cyano, nitro, hydroxyl, substituted or unsubstituted amino, substituted or unsubstituted carbonyl, substituted or unsubstituted C₁₋₆ alkyl, substituted or unsubstituted C₁₋₆ alkoxy, substituted or unsubstituted C₁₋₆ alkenyl, substituted or unsubstituted C₆₋₁₀ aryl, or substituted or unsubstituted heterocyclyl;
m1 and m2 are independently 1, 2, or 3; n1 and n2 are independently 0, 1, or 2; and p1 and p2 are independently 0, 1, or 2;
wherein if X₁ is O or S, then R₁ is absent, and wherein if X₂ is O or S, then R₆ is absent;
or a pharmaceutically acceptable salt, tautomer, stereoisomer, or prodrug thereof, and a pharmaceutically acceptable excipient.

15. The method of claim 13 wherein the treatment is for the prevention or remission of cardiotoxicity.

16. The method of claim 13 further comprising the administration of a cancer chemotherapeutic agent.

17. The method of claim 15 wherein the cancer chemotherapeutic agent is an EGFR modulating agent or an anthracycline compound.

18. The method of claim 16 wherein the cancer chemotherapeutic agent is selected from the group consisting of trastuzumab, cetuximab, bevacizumab, panitumumab, gefitinib, erlotinib, or combinations thereof.

19. The method of claim 17 wherein the cancer chemotherapeutic agent is selected from the group consisting of daunorubicin, doxorubicin, epirubicin, idarubicin, and combinations thereof.

20. The method of claim 13 wherein the compound is dexrazoxane.
